(19) United States
(12) Patent Application Publication
(10) Pub. No.: US 2011/0311651 A1
(43) Pub. Date: Dec. 22, 2011

(54) CARDENOLIDES FOR THE TREATMENT OF OCULAR CANCER

(75) Inventors: Hakim Djaballah, Scarsdale, NY (US); Christophe Antczak, New York, NY (US); David H. Abramson, New York, NY (US)

(73) Assignee: Sloan-Kettering Institute for Cancer Research, New York, NY (US)

(21) Appl. No.: 13/132,706

(22) PCT Filed: Dec. 2, 2009
(86) PCT No.: PCT/US09/06360

§ 371 Date: Aug. 8, 2011

Related U.S. Application Data
(60) Provisional application No. 61/121,401, filed on Dec. 10, 2008.

(51) Int. Cl. A61K 33/24; A61K 31/7048; C12N 5/09; A61M 37/00; A61K 38/13; A61P 35/00; A61P 35/04; A61K 31/585; A61K 31/593

(52) U.S. Cl. 424/649; 514/175; 514/26; 435/375; 514/27; 514/167; 514/19.9; 600/1; 604/20

(57) ABSTRACT

The instant invention provides methods of using a class of compounds known as cardenolides in the treatment of proliferative diseases such as cancer. In particular, the instant invention provides methods of treating ocular cancer {e.g., retinoblastoma) using intraarterial infusion to administer cardenolides locally to the eye of a subject with an ocular cancer.

### RELATED APPLICATIONS

The present application claims priority under 35 U.S.C. §119(e) to U.S. provisional application, U.S. Ser. No. 61/121,401, filed Dec. 10, 2008, which is incorporated herein by reference.

### BACKGROUND OF THE INVENTION

Retinoblastoma (Rb) is an ocular cancer that affects approximately 5,000 to 8,000 children worldwide each year and constitutes the most common primary ocular tumor of childhood (Abramson, Invest. Ophthalmol. Vis. Sci. 2005, 46, 2683-2691). Retinoblastoma occurs in a germline (40%) and non-germline (60%) form and results from the loss of function of both alleles of the retinoblastoma tumor suppressor gene in retinal progenitor cells (Cavenee et al., Science 1985, 228, 501-503; Friend et al., Nature 1986, 323, 643-646; Godbout et al., Nature 1983, 304, 451-453). Because the primary role of the retinoblastoma pathway is to regulate cell proliferation, loss of Rb1 plays an important role in cancer development in ocular cancers as well as nonocular cancers. Indeed, the presence of a defective gene predisposes patients to develop multiple ocular and nonocular tumors and further complicates treatment.

The current survival rate associated with retinoblastoma is approximately 90% in developing countries (De Potter et al., Curr. Opin. Ophthalmol. 2002, 13, 331-336); however, effective treatment often requires removal of the affected eye (enucleation). Alternative treatments do exist, and multimodal treatment approaches are typically pursued by ocular oncologists either in combination with enucleation or in an attempt to salvage one or both eyes with advanced disease. Exemplary combinations of multimodal treatment regimens include the use of external beam therapy and/or chemotherapy employed to shrink the tumor prior to local treatment such as thermotherapy, cryotherapy, and brachytherapy (Wilson, et al., Investigative Ophthalmology & Visual Science, 2006, 47(4), 1269-1273). Unfortunately, complications from the use of multimodal treatment approaches can be severe and are particularly harmful to young children.

Complications from radiotherapy and systemic chemotherapy are especially common in children genetically predisposed to tumor development, and the long term effects of external beam radiotherapy can include cataracts, radiation retinopathy, impaired vision, temporal bone suppression (De Potter et al., Curr. Opin. Ophthalmol. 2002, 13, 331-336), and an increased incidence of secondary cancers, particularly for children under the age of one. Side-effects of systemic chemotherapy include cytopenia, neutropenia, gastrointestinal distress, and neurotoxicity (Brichard et al., Med. Pediatr. Oncol. 2002, 38, 411-415; Benz et al., Arch. Ophthalmol. 2000, 118, 577-578; Beck et al., J. Clin. Oncol. 2000, 18, 2881-2887; Friedman et al., J. Clin. Oncol. 2000, 18, 12-17; Rizzuti et al., Arch. Ophthalmol. 2008, 126, 862-865). Also, the use of platinum-based drugs for the treatment of childhood malignancies increases a child's risk of developing secondary malignancies, and the use of etoposide, a topoisomerase II inhibitor, has been linked to the development of secondary leukemias (Klein et al., Eur. J. Cancer 2003, 39, 808-817; Nishimura et al., J. Clin. Oncol. 2001, 19, 4182-4183; Chantada et al., Med. Pediatr. Oncol. 2003, 40, 158-161). Consequently, available treatments for retinoblastoma are limited and often devastating, necessitating the development of new, more effective alternatives.

### SUMMARY OF THE INVENTION

Retinoblastoma treatment regimens incorporating chemotherapy stand to benefit substantially from improved drug delivery of the chemotherapeutic agent into the ophthalmic artery. One such treatment method being pursued is direct intraarterial infusion (Abramson et al., Ophthalmology 2008, 115(8), 1398-1404). Intraarterial infusion delivers the drug locally and provides a promising new approach to chemotherapy by preventing toxic agents from entering the systemic circulation. Minimizing systemic exposure affords many drugs diminished toxicity and improved efficacy to the extent that reinvestigation of previously unsuitable chemotherapeutic agents may now lead to new treatment options when combined with intraarterial infusion. Thus, it is important to reconsider the utility of known biologically active compounds that were previously dismissed for their undesirable toxicity profiles.

The instant invention describes the results of an investigation aimed at identifying chemotherapeutic agents to be administered via direct intraarterial infusion for the treatment of retinoblastoma. Potent agents for treating retinoblastoma were identified among a library of 2,640 compounds consisting of marketed drugs, bioactive compounds in various therapeutic areas, toxic substances, and natural products. Importantly, it was found that the newly identified agents for treating retinoblastoma belong to a well-described pharmacological classes, some agents currently being used in clinic for other purposes. Of the candidates identified, cardenolides proved particularly efficacious in treating retinoblastoma. Cardenolides are a well-defined class of compounds previously used for the treatment of cardiovascular disease. The utility of these compound in the treatment of retinoblastoma via intraarterial infusion of the drug into the ophthalmic artery is described herein.

In one aspect, the invention provides a method of treating a subject with an ocular cancer comprising administering a therapeutically effective amount of a cardenolide locally to the eye with the cancer. In some embodiments, the cardenolide is of the formula:

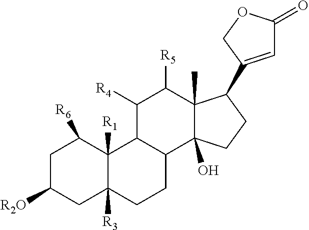

wherein:

R₁ is H, OH, CH₃, CH₂OH, or CHO;

R₂ is H or a carbohydrate moiety;

R₃ is H or OH;

R₄ is H or OH;

R₅ is H or OH;

R₆ is H or OH;

or a pharmaceutically acceptable form thereof. In some embodiments, the cardenolide is selected from the group consisting of digoxigenin, ouabain, neriifolin, digoxin, acetyldigoxin, peruvoside, digitoxin, acetyldigitoxins, digitoxigenin, medigoxin, strophanthins, cymarin, and strophanthidin.

In certain embodiments, R₂ is H. In some embodiments, R₂ is a carbohydrate moiety. In certain embodiments, R₂ is a glycoside. In certain embodiments, R₂ is a starch, glycogen, dextran, cyclodextran, or hyaluranic acid. In some embodiments, R₂ is an oligosacharide. In certain embodiments, R₂ is a disaccharide such as sucrose, lactose, or maltose. In some embodiments, R₂ may be a monosaccharide such as glucose, fructose, galactose, mannose, xylose, or ribose. In some embodiments, R₂ is a carbohydrate derivative (e.g., an ester, ether, aminated, amidated, sulfated, phosphosubstituted, or otherwise suitably protected carbohydrate. (e.g., an ester, ether, aminated, amidated, sulfated, phosphosubstituted, or otherwise suitably protected carbohydrate).

In some embodiments, the cardenolide is of the formula:

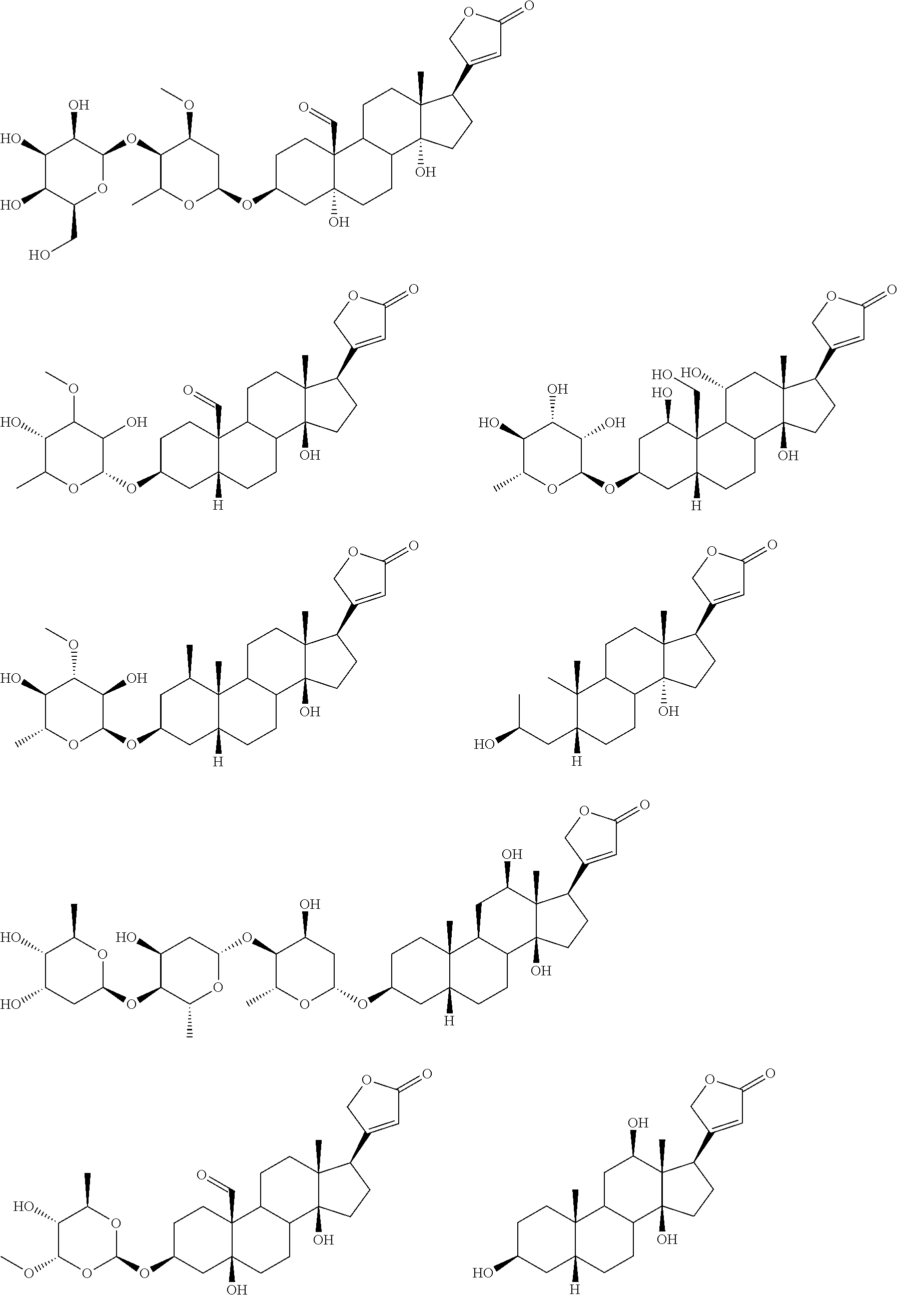

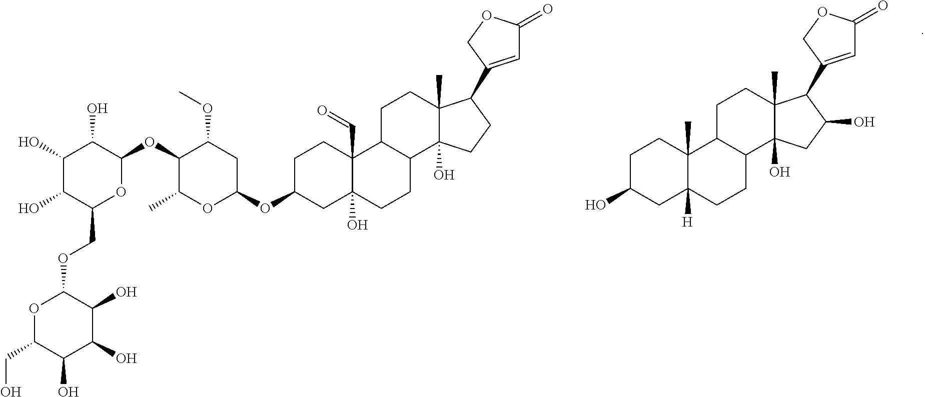

In certain embodiments, the instant invention provides a method of treating ocular cancers wherein the cardenolide is digoxin. Exemplary ocular cancers include, but are not limited to, retinoblastoma (Rb), medulloepithelioma, ocular melanoma, lymphoma, or other cancers which have metastasized to the eye. In some embodiments, the ocular cancer is retinoblastoma. In some embodiments, the method further comprises administering a second type of therapy. In certain embodiments, the second type of therapy comprises administration of a second chemotherapeutic agent. In certain embodiments, the second type of therapy is a type of radiation therapy. In another aspect, the instant invention provides a method of treating an ocular cancer comprising administering a therapeutically effective amount of digoxin via direct intraarterial infusion into the ophthalmic artery of the eye of a subject with ocular cancer. In certain embodiments, the subject is a human. Exemplary ocular cancers that may be treated by local administration of digoxin include, but are not limited to, ocular cancers such as retinoblastoma, medulloepithelioma, ocular melanoma, lymphoma, or other cancer which has metastasized to the eye. In certain embodiments, the cancer is retinoblastoma.

In another aspect, the instant invention provides a method of inhibiting the growth of ocular cancer cells comprising contacting in vitro ocular cancer cells with an effective amount of a cardenolide to inhibit the growth of the cells. In some embodiments, inhibiting the growth of ocular cancer cells further comprises using a second chemotherapeutic agent in combination with the cardenolide. In certain embodiments, the cells are derived from a retinoblastoma tumor. In certain embodiments, the cells are human retinoblastoma cells Y79, WERI-Rb-1, RB355, or Y79LUC.

In certain embodiments, the instant invention provides a pharmaceutical composition for treating an ocular cancer for local administration comprising a cardenolide and a pharmaceutically acceptable excipient. Exemplary ocular cancers include, but are not limited to, retinoblastoma, medulloepithelioma, ocular melanoma, lymphoma, or other cancers which have metastasized to the eye. In certain embodiments, the cancer is retinoblastoma. In some embodiments, the compounds are administered via direct intraarterial infusion. In certain embodiments, the compounds are administered via direct intraarterial infusion into the ophthalmic artery of the eye of a subject with one of the above-mentioned cancers. In certain embodiments, the compounds are administered via direct intraarterial infusion into the ophthalmic artery of the eye of a human under the age of 18 with one of the above-mentioned cancers.

In some embodiments, the instant invention provides methods for treating cancer by locally administering a cardenolide and a pharmaceutically acceptable exipient to a subject in need thereof. In certain embodiments, local administration comprises intraarterial infusion, and the cancer is hematopoietic, liposarcoma, lung, brain, liver, or pancreatic cancer. In certain embodiments, the pharmaceutical composition comprises the cardenolide digoxin. In certain embodiments, the subject is human.

All publications and patent documents cited in this application are incorporated by reference in their entirety for all purposes to the same extent as if the contents of each individual publication or patent document were incorporated herein.

### Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic, aliphatic and heteroaliphatic, carbon and heteroatom substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative diseases, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

Certain compounds of the present invention can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds.

Where a particular enantiomer is desired, it may, in some embodiments be provided substantially free of the corresponding enantiomer, and may also be referred to as “optically enriched.” “Optically enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 96%, 97%, 98%, or 99% by weight of a desired enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

The term “acyl”, as used herein, refers to a carbonyl-containing functionality, e.g., —C(═O)R′, wherein R′ is an aliphatic, alycyclic, heteroaliphatic, heterocyclic, aryl, heteroaryl, (aliphatic)aryl, (heteroaliphatic)aryl, heteroaliphatic(aryl), or heteroaliphatic(heteroaryl) moiety, whereby each of the aliphatic, heteroaliphatic, aryl, or heteroaryl moieties is substituted or unsubstituted, or is a substituted (e.g., hydrogen; or aliphatic, heteroaliphatic, aryl, or heteroaryl moieties) oxygen or nitrogen containing functionality (e.g., forming a carboxylic acid, ester, or amide functionality).

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted or unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl(propargy1), 1-propynyl, and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure —NH₂R′, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino, and the like.

Some examples of substituents of the above-described aliphatic (and other) moieties include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)R_{x;} wherein each occurrence of Rₓ independently includes, but is not limited to, aliphatic, alycyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the term “aryl”, as used herein, refers to a stable mono- or polycyclic, unsaturated moiety having preferably 3-14 carbon atoms, each of which may be substituted or unsubstituted. In certain embodiments, the term “aryl” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaryl”, as used herein, refers to a stable mono- or polycyclic, unsaturated moiety having preferably 3-14 carbon atoms, each of which may be substituted or unsubstituted; and comprising at least one heteroatom selected from O, S, and N within the ring (i.e., in place of a ring carbon atom). In certain embodiments, the term “heteroaryl” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aryl and heteroaryl moieties, as defined herein may be attached via an alkyl or heteroalkyl moiety and thus also include -(alkyl)aryl, -(heteroalkyl)aryl, -(heteroalkyl)heteroaryl, and -(heteroalkyl)heteroaryl moieties. Thus, as used herein, the phrases “aryl or heteroaryl moieties” and “aryl, heteroaryl, -(alkyl)aryl, -(heteroalkyl)aryl, -(heteroalkyl)heteroaryl, and -(heteroalkyl)heteroaryl” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl, and the like.

The term “heteroaryl”, as used herein, does not differ significantly from the common meaning of the term in the art and refers to a cyclic aromatic radical having from five to ten ring atoms of which one ring atom is selected from S, O, and N; zero, one or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl, or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated that any two adjacent groups taken together may represent a 4, 5, 6, or 7-membered substituted or unsubstituted alicyclic or heterocyclic moiety. Additional examples of generally applicable substituents are illustrated by the specific embodiments described herein.

The term “cycloalkyl”, as used herein, refers specifically to groups having three to seven, preferably three to ten, carbon atoms. Suitable cycloalkyls include, but are not limited to cyclopropyl, cyclobutyl, cyclopentyl, cyclohexyl, cycloheptyl, and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)R_{x;} wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated o runsaturated. In certain embodiments, heteroaliphatic moieties are substituted by independent replacement of one or more of the hydrogen atoms thereon with one or more moieties including, but not limited, to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments described herein.

The term “heterocycloalkyl”, “heterocycle,” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S, and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S, and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two, or three of the hydrogen atoms thereon with, but are not limited to, aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substitutents described above and herein may be substituted or unsubstituted. Additional examples or generally applicable substituents are illustrated by the specific embodiments described herein.

The terms “carbohydrate” as used herein refers to compounds of the general molecular formula CₙH₂ₙOₙ. Most carbohydrates are aldehydes or ketones with multiple hydroxyl groups, usually one on each carbon atom of the molecule. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

Additionally, it will be appreciated that any of the alicyclic or heterocyclic moieties described above and herein may comprise an aryl or heteroaryl moiety fused thereto. Additional examples of generally applicable substituents are illustrated by the specific embodiments described herein. The terms “halo” and “halogen” as used herein refer to an atom selected from fluorine, chlorine, bromine, and iodine.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}), or quaternary (—N⁺RₓR_{y}R_{z}) amine, where Rₓ, R_{y} and R_{z} are independently an aliphatic, alicyclic, heteroaliphatic, heterocyclic, aryl, or heteroaryl moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “alkylidene”, as used herein, refers to a substituted or unsubstituted, linear or branched saturated divalent radical consisting solely of carbon and hydrogen atoms, having from one to n carbon atoms, having a free valence “-” at both ends of the radical. In certain embodiments, the alkylidene moiety has 1 to 6 carbon atoms.

The term “alkenylidene”, as used herein, refers to a substituted or unsubstituted, linear or branched unsaturated divalent radical consisting solely of carbon and hydrogen atoms, having from two to n carbon atoms, having a free valence “-” at both ends of the radical, and wherein the unsaturation is present only as double bonds and wherein a double bond can exist between the first carbon of the chain and the rest of the molecule. In certain embodiments, the alkenylidene moiety has 2 to 6 carbon atoms.

The term “alkynylidene”, as used herein, refers to a substituted or unsubstituted, linear or branched unsaturated divalent radical consisting solely of carbon and hydrogen atoms, having from two to n carbon atoms, having a free valence “-” at both ends of the radical, and wherein the unsaturation is present only as triple or double bonds and wherein a triple or double bond can exist between the first carbon of the chain and the rest of the molecule. In certain embodiments, the alkynylidene moiety has 2 to 6 carbon atoms.

Unless otherwise indicated, as used herein, the terms “alkyl”, “alkenyl”, “alkynyl”, “heteroalkyl”, “heteroalkenyl”, “heteroalkynyl”, “alkylidene”, alkenylidene“, -(alkyl)aryl, -(heteroalkyl)aryl, -(heteroalkyl)aryl, -(heteroalkyl)heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. The biological activity of pro-drugs and pro-drugs may also be altered by appending a functionality onto the compound, which may be catalyzed by an enzyme. Also, included are oxidation and reduction reactions, including enzyme-catalyzed oxidation and reduction reactions. Certain exemplary pharmaceutical compositions and pharmaceutically acceptable derivatives will be discussed in more detail herein below.

By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis, in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As used herein, the term “tautomer” includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol; amide-to-imide; lactam-to-lactim; enamine-to-imine; and enamine-to-(a different) enamine tautomerizations.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

“Compound”: The term “compound” or “chemical compound” as used herein can include organometallic compounds, organic compounds, metals, transitional metal complexes, and small molecules. In certain preferred embodiments, polynucleotides are excluded from the definition of compounds. In other preferred embodiments, polynucleotides and peptides are excluded from the definition of compounds. In a particularly preferred embodiment, the term compounds refers to small molecules (e.g., preferably, non-peptidic and non-oligomeric) and excludes peptides, polynucleotides, transition metal complexes, metals, and organometallic compounds.

“Small Molecule”: As used herein, the term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 2000 g/mol, preferably less than 1500 g/mol, although this characterization is not intended to be limiting for the purposes of the present invention. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In certain other preferred embodiments, natural-product-like small molecules are utilized.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human subject affected by a condition or disease to be diagnosed or investigated).

The biological sample can be in any form, including a solid material such as a tissue, cells, a cell pellet, a cell extract, cell homogenates, or cell fractions; or a biopsy, or a biological fluid. The biological fluid may be obtained from any site (e.g., blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g., fluid obtained from an abscess or any other site of infection or inflammation), or fluid obtained from a joint (e.g., a normal joint or a joint affected by disease such as rheumatoid arthritis, osteoarthritis, gout or septic arthritis).

The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates, and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### BRIEF DESCRIPTION OF THE DRAWINGS

FIG. 1. Heat map comparative analysis of the Y79 and RB355 cell line screens. The percentage inhibition for each tested compound in both screens is represented as a heat map.

FIG. 2. The Scatter plot comparative analysis of the Y79 and RB355 screens. The percentage inhibition for each tested compound in both screens is represented as a scatter plot. The 29 positives at a threshold of 90% inhibition in both screens are highlighted in red, and the 19 cardenolides present in the library are highlighted in green.

FIG. 3. Summary of the structures for the 11 positives selected at a threshold of 95% inhibition in both screens. (A) Cardenolides. (B) Non-cardenolides.

FIG. 4. Structure-activity relationship study for a collection of 35 cardenolides in a panel of four ocular cancer cell lines: RB355, C918, Y79 and WERI-Rb-1 (A) Heat map and numerical summary of calculated IC₅₀s for the 35 cardenolides in the ocular cancer cell line panel. The structure of identified chemical scaffolds is highlighted. (B) Representative dose response curves generated for the cardenolide SKI 343995 in the panel of ocular cancer cell lines. (C) Representative dose response curves generated for the drug ouabain in the panel of ocular cancer cell lines.

FIG. 5. Compared potency of the drug ouabain against (A) Y79 cells and (B) RB355 cells with the clinical agents vincristine, etoposide, carboplatin, cisplatin and the experimental drugs nutlin-3 and calcitriol. (C) Summary of the calculated IC₅₀s.

FIG. 6. Immunofluorescence detection of activated Caspase-3 in Y79 cells treated with (A) 1% DMSO (v/v); (B) 100 μM vincristine, 1% DMSO (v/v); (C) 10 μM etoposide, 1% DMSO (v/v); and (D) 0.5 μM ouabain, 1% DMSO (v/v).

FIG. 7. In vivo antitumor effect of the drug ouabain evaluated by bioluminescent imaging of tumor burden in a mouse xenograft model of retinoblastoma. Images of a representative mouse from each group treated with either vehicle only (10% DMSO v/v) or 15 mg/kg ouabain in 10% DMSO v/v over 19 days are shown.

FIG. 8. (A) In vivo antitumor effect of the drug ouabain evaluated by tumor volume measurement in a mouse xenograft model of retinoblastoma. The average tumor volume per group over 19 days treatment is plotted. (B) Monitoring of animal weight. The average animal weight per group over 19 days treatment is plotted.

FIG. 9. Summary of the eleven positives identified in the RB355/Y79 screening campaign. Positives belonging to the class of cardenolides are highlighted in orange. The calculated IC₅₀s for each positive in the ocular cancer cell line cytotoxicity panel are detailed.

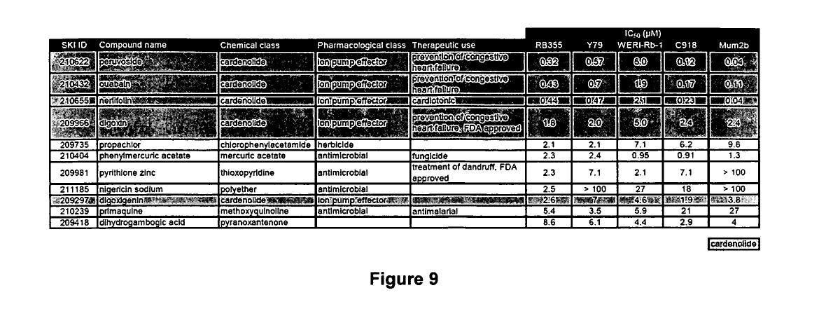

FIG. 10. In vivo antitumor effect of the drug ouabain evaluated by bioluminescent imaging of tumor burden in a mouse xenograft model of retinoblastoma. Images of a representative mouse treated with Ouabain (1.5 mg/Kg minipump weekly X 4) in 10% DMSO over 19 days are shown.

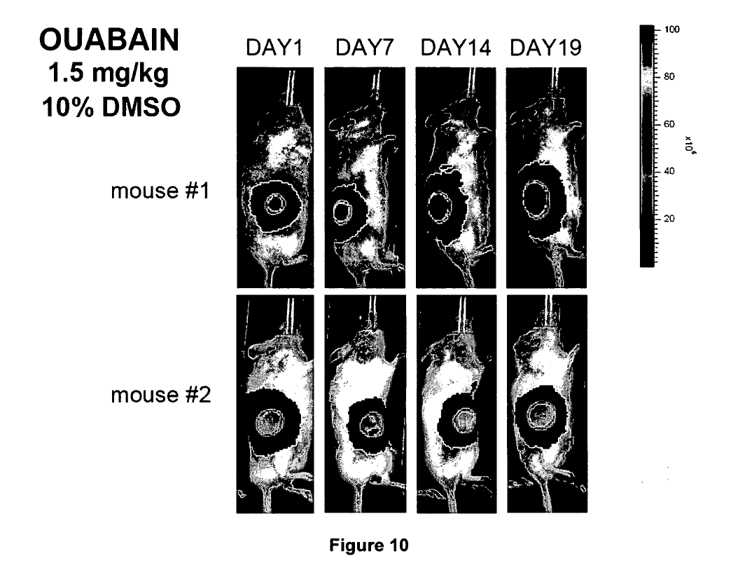

FIG. 11. In vivo antitumor effect of the drug ouabain evaluated by bioluminescent imaging of tumor burden in a mouse xenograft model of retinoblastoma. Images of a representative mouse treated with Ouabain (15 mg/Kg minipump weekly×4) in 10% DMSO over 19 days are shown.

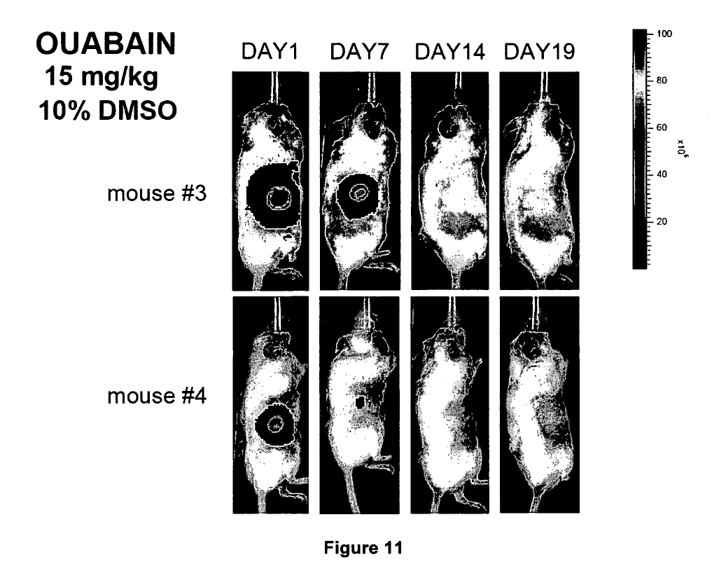

FIG. 12. Images of a representative mouse treated with 10% DMSO (minipump weekly×4) over 19 days are shown.

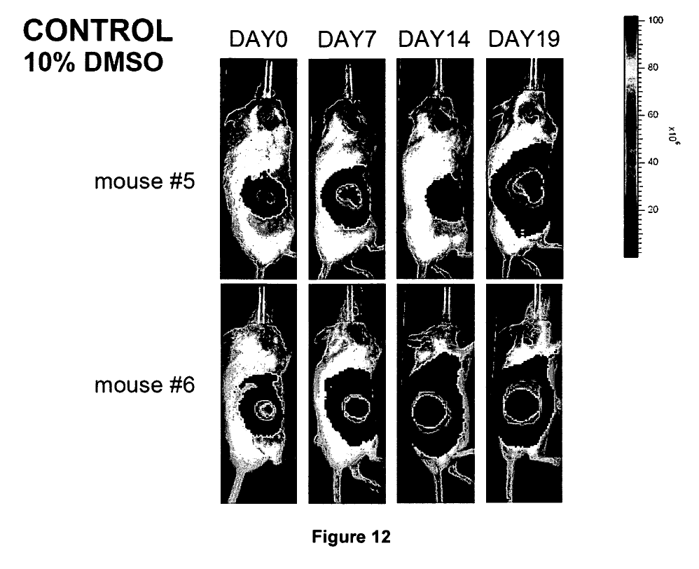

### DETAILED DESCRIPTION OF THE INVENTION

Ocular cancer can be a difficult and devastating disease to treat, often resulting in the partial or total loss of vision due to enucleation or other therapies. Retinoblastoma is the most common childhood primary intraocular malignancy and affects approximately 5,000 to 8,000 children worldwide each year. Until the 1990's, enucleation and/or external beam radiotherapy (EBRT) were the standards of care for children with advanced bilateral disease. However, an increased incidence of secondary malignancies as well as disfigurement caused by orbital hypoplasia associated with scatter doses of radiation has spurred investigators to seek alternative forms of treatment. Accordingly, regimens incorporating systemic chemotherapy as a means of shrinking the tumor prior to focal treatments has replaced EBRT as the primary treatment for retinoblastoma. Unfortunately, the severe toxicity associated with systemic chemotherapy, especially in children under the age of one, has limited the efficacy of this method of treatment and has led to the development of improved approaches for treating patients with retinoblastoma. One such approach involves localized drug delivery of the chemotherapeutic agent via direct intraarterial infusion into the ophthalmic artery. Direct intraarterial infusion when applied to the treatment of retinoblastoma involves the delivery of high-dose chemotherapy via ophthalmic artery infusion. This prevents the drug from entering the systemic bloodstream, substantially reducing systemic toxicity. Thus, direct intraarterial infusion through the ophthalmic artery not only allows for higher doses of current chemotherapeutic agents for ocular cancers, but also warrents reconsideration of previously dismissed drug candidates whose toxicity profiles proved incompatible with conventional systemic administration.

The present invention describes the first chemical screen aimed specifically at identifying alternative chemotherapeutic agents for local delivery in the treatment of retinoblastoma. Among a library of 2,640 compounds consisting of marketed drugs, bioactive compounds in various therapeutic areas, natural products, and toxic substances, several potent agents were identified based on cytotoxicity. One particularly potent class of compounds identified in this screen are also used in the treatment of heart disease. They are known as the cardenolides. Given their current use in the clinic, many cardenolides are well-characterized and have well-defined pharmacological profiles. Thus, they provide attractive candidates for use in the treatment of retinoblastoma, particularly the local administration of a cardenolide into the ophthalmic artery.

### Compounds of the Invention

In part, the present invention encompasses the recognition that a compound of the formula shown below may be used to treat an ocular cancer when administered locally to the eye of a subject with an ocular cancer. In general, the present invention provides a method of treating an ocular cancer comprising administering a therapeutically effective amount of a cardenolide locally to the eye of a subject with an ocular cancer, wherein the cardenolide is of the formula:

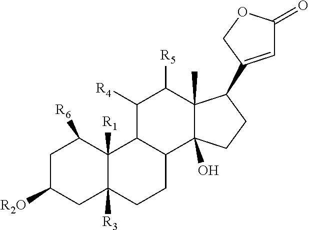

wherein:

R₁ is H, OH, CH₃, CH₂OH, or CHO;

R₂ is H or a carbohydrate moiety;

R₃ is H or OH;

R₄ is H or OH;

R₅ is H or OH;

R₆ is H or OH;

or a pharmaceutically acceptable form thereof.

In some embodiments, R₁ is H. In some embodiments, R₁ is OH. In some embodiments, R₁ is CH₃. In some embodiments, R₁ is CH₂OH. In some embodiments, R₁ is CHO.

In some embodiments, R₂ is H. In some embodiments, R₂ is a carbohydrate moiety. In certain embodiments, R₂ is a glycoside. In certain embodiments, R₂ is a starch, glycogen, dextran, cyclodextran, or hyaluranic acid. In some embodiments, R₂ is an oligosacharide. In certain embodiments, R₂ is a disaccharide such as sucrose, lactose, or maltose. In some embodiments, R₂ may be a monosaccharide such as glucose, fructose, galactose, mannose, xylose, or ribose. In some embodiments, R₂ is a carbohydrate derivative (e.g., an ester, ether, aminated, amidated, sulfated, phosphosubstituted, or otherwise suitably protected carbohydrate).

In some embodiments, R₃ is H. In some embodiments, R₃ is OH.

In some embodiments, R₄ is H. In some embodiments, R₄ is OH.

In some embodiments, R₅ is H. In some embodiments, R₅ is OH.

In some embodiments, R₆ is H. In some embodiments, R₆ is OH.

In certain embodiments, the cardenolide is any one of the formulae:

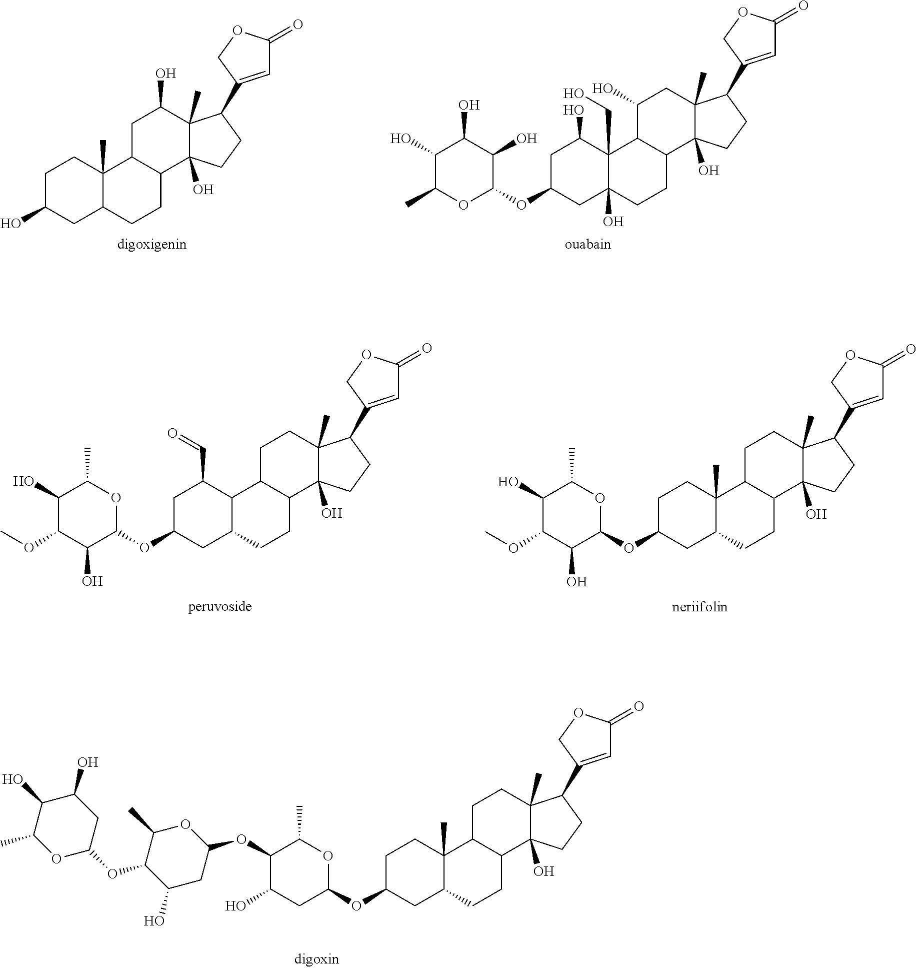

In certain embodiments, the cardenolide is digoxin.

In certain embodiments, the cardenolide is ouabain.

In certain embodiments, the inventive method comprises administering a therapeutically effective amount of a cardenolide locally to the eye of a subject with an ocular cancer, wherein the cardenolide is selected from the group of previously known cardenolides. In certain embodiments, the cardenolide is digoxigenin. In certain embodiments, the cardenolide is ouabain. In certain embodiments, the cardenolide is neriifolin. In certain embodiments, the cardenolide is digoxin. In certain embodiments, the cardenolide is acetyldigoxin. In certain embodiments, the cardenolide is peruvoside. In certain embodiments, the cardenolide is digitoxin. In certain embodiments, the cardenolide is acetyldigitoxin. In certain embodiments, the cardenolide is digitoxigenin. In certain embodiments, the cardenolide is medigoxin. In certain embodiments, the cardenolide is strophanthins. In certain embodiments, the cardenolide is cymarin. In certain embodiments, the cardenolide is strophanthidin.

Some of the foregoing compounds include one or more asymmetric centers, and thus can exist in various isomeric forms, e.g., stereoisomers and/or diastereomers. Thus, compounds useful in the present invention and pharmaceutical compositions thereof may be in the form of an individual enantiomer, diastereomer, or geometric isomer, or may be in the form of a mixture of stereoisomers. In certain embodiments, the compounds utilized in the invention are enantiopure compounds. In certain other embodiments, mixtures of stereoisomers or diastereomers are utilized.

The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these cardenolides and compositions comprising one or more cardenolides and one or more pharmaceutically acceptable excipients or additives.

Compounds utilized in the invention may be prepared by crystallization of the compound under different conditions and may exist as one or a combination of polymorphs of the compound. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram, and/or other techniques. Thus, the present invention encompasses cardenolides, their derivatives, their tautomers, their pro-drugs, their stereoisomers, their polymorphs, their pharmaceutically acceptable salts, their pharmaceutically acceptable solvates, their pharmaceutically acceptable hydrates, their pharmaceutically acceptable co-crystals, and pharmaceutically acceptable compositions thereof.

### Methods of Treatment

The instant invention provides methods of administering a therapeutically effective amount of a cardenolide locally to the eye of a subject with an ocular cancer. Local administration offers an improved approach to drug delivery because it allows the cytotoxic drug to bypass the systemic circulation and thereby minimizes the systemic toxicity of chemotherapeutic agents. Lower toxicity profiles allow for higher, more effective doses to be administered to the subject, potentially improving a subject's prognosis.

Local administration of a cardenolide as described herein to the eye of a subject with an ocular cancer allows for improved efficacy and/or decreased toxicity. In some embodiments, the subject is a mammal. In some embodiments, the subject is a rodent. In some embodiments, the subject is a rat. In some embodiments, the subject is a mouse. In certain embodiments, the subject is a human. In certain embodiments, the subject is a human less than 18 years of age. In certain embodiments, the subject is a human less than 10 years of age. In certain embodiments, the subject is a human less than 5 years of age. In certain embodiments, the subject is a human less than 2 years of age. In certain embodiments, the subject is less than one year of age. In certain embodiments, the subject is 1, 2, 3, 4, 5, 6, 7, 8, 9, or 10 years of age.

The ocular cancer being treated using the inventive treatment may be any one of a number of ocular cancers. In some embodiments, the ocular cancer is retinoblastoma. In certain embodiments, the ocular cancer is advanced retinoblastoma. In certain embodiments, the ocular cancer is advanced bilateral retinoblastoma. In certain embodiments, the retinoblastoma is intraocular retinoblastoma. In certain embodiments, the retinoblastoma is extraocular retinoblastoma. In certain embodiments, the retinoblastoma is recurrent retinoblastoma. In some embodiments, the cancer is medulloepithelioma. In some embodiments, the cancer is a ocular melanoma. In some embodiments, the cancer is lymphoma. In some embodiments, the cancer is a cancer that has metastasized to the eye.

The treatment may include a second therapy. The second therapy may be chemotherapy, radiotherapy, cryotherapy, external beam radiotherapy, thermotherapy, or brachytherapy. In certain embodiments, the second type of therapy used with local administration of a cardenolide is external beam radiotherapy. In certain embodiments, the second type of therapy is administration of a second chemotherapeutic agent, for instance, etoposide, teniposide, or verapamil. In some embodiments, the second chemotherapeutic agent is vincristine, calcitriol, melphalan, 5-fluorouracil, cyclosporin, carboplatin, cisplatin, topotecan or Nutlin-3. In certain embodiments, the second chemotherapeutic is an inhibitor of MDR1/Pgp. In some embodiments, the second chemotherapeutic agent is a multidrug resistance-associated protein-1 (MRP1) inhibitor or an ABC transporter inhibitor. In certain embodiments, the second chemotherapeutic agent is any one of those described herein and the cardenolide is digoxin, acetyldigoxins, ouabain, neriifolin, digoxigenin, peruvoside, digitoxin, acetyldigitoxins, digitoxigenin, medigoxin, strophanthins, cymarin, or strophanthidin. In some embodiments, the second agent is delivered locally. In some embodiments, the second agent is delivered systemically.

In some embodiments, the cardenolide is administered locally to the site of the disease. In some embodiments, local administration comprises local intraarterial infusion. In certain embodiments, local administration comprises direct intraarterial infusion into an artery that delivers blood to the diseased site. In some embodiments, local administration comprises direct intraarterial infusion into an ophthalmic artery, and the disease is an ocular cancer. In certain embodiments, direct intraarterial infusion into an ophthalmic artery of a subject with ocular cancer includes performing an arteriogram in order to visualize the vasculature around a desired artery, cathetering the desired artery, optionally performing a second angiogram to confirm that the desired artery vascularizes the diseased site, and infusing the artery with a therapeutically effective amount of chemotherapeutic agent, wherein the agent is a cardenolide. In certain embodiments, a cardenolide and carboplatin are administered as a combination therapy.

The time of infusion ranges from approximately 1 minute to approximately 120 minutes. In some embodiments, the time of infusion ranges from approximately 1 minute to approximately 90 minutes. In some embodiments, the time of infusion ranges from approximately 1 minute to approximately 60 minutes. In some embodiments, the time of infusion ranges from approximately 5 minutes to approximately 45 minutes. In some embodiments, the time of infusion ranges from approximately 15 minutes to approximately 45 minutes. In some embodiments, the time of infusion is approximately 30 minutes. In certain embodiments, the treatment is repeated at least two times. In certain embodiments, the treatment is repeated at least three times. In certain embodiments, the treatment is repeated at least four times. In certain embodiments, the treatment is repeated 2, 3, 4, 5, 6, 7, 8, 9, 10 times. In some embodiments, the treatment is repeated upon recurrence.

The efficacy of the inventive treatment may be evaluated using any method known in the art. The treatment of the cancer may be evaluated by physical examination, laboratory testing, imaging studies, electrophysiological studies, etc. Exemplary methods include external examination, visual acuity testing, pupil and motility evaluation, complete fundus examination under anesthesia including RetCam digital photography, and standard electroretinogram testing under photopic and scotopic conditions. In some embodiments, evaluation may include systemic evaluations comprising interval medical history, weight and height measurements, and complete blood counts. In some embodiments, the efficacy of the inventive treatment is evaluated using any combination of methods known in the medical arts.

### Method of Inhibiting Growth

Methods of assaying compounds are needed to determine a compound's efficacy in treating different types of cancer. The inhibition of the growth of ocular cells may be determined in vivo or in vitro. The instant invention provides methods of inhibiting the growth of ocular cells comprising contacting in vitro ocular cancer cells with an effective amount of a cardenolide to inhibit the growth of cells. This method may comprise steps of contacting a cardenolide with cells, and then incubating the cells under suitable conditions to test for the inhibition of growth, and at various concentrations to fully determine the extent of cell growth.

The cells may be derived from a cancer cell line or a biological sample (e.g., a biopsy). Any cells found in the eye may be used in the inventive method. In certain embodiments, the cells are normal cells. In certain embodiments, the cells are cancer cells. In certain embodiments, the cancer cells are ocular cancer cells such as retinoblastoma cells. In certain embodiments, the ocular cancer cell line is the human retinoblastoma cell line Y79, WERI-Rb-1, RB355, or Y79LUC. In some embodiments, the ocular cancer cell line is a melanoma cell line. In certain embodiments, the ocular cancer is a uveal melanoma cell line such as C918 or Mum2b. In certain embodiments, the cells are derived from a biopsy of a patient with an ocular cancer. Such cells may be tested in vitro to determine the efficacy of a cardenolide on the patient's cancer cells.

### Methods of Screening for Inventive Compounds

The instant invention also provides methods of identifying compounds that are useful in treating retinoblastoma, particularly those that may be useful in local delivery of a the compound to a diseased site. Such methods were used to identify compounds that act as antiproliferative agents from a large collection of chemical compounds as described below in the Examples. In certain embodiments, the instant invention provides methods of identifying compounds which inhibit cellular proliferation. In certain embodiments, the instant invention provides methods of evaluating the potency of a test compound against a particular type of cell. In some embodiments, the instant invention provides methods of assessing the in vivo efficacy of a test compound against a particular cancer. In certain embodiments, the inventive methods are high-throughput methods. For example, hundreds or thousands of compounds may be evaluated in parallel. In some embodiments, in order to identify chemical scaffolds with broad cytotoxicity toward retinoblastoma, libraries of compounds can be screened against two different cell lines in parallel.

In certain embodiments, the instant invention provides a method of screening a library of compounds based on a cytotoxicity assay. In some embodiments, the instant invention includes a method of identifying compounds which inhibit cellular proliferation. This method comprises the steps of first providing library compounds of interest, contacting said library compounds with cells of interest, and incubating the compounds and cells of interest under suitable conditions. The antiproliferative activity of the compounds of interest can be assessed using any of the methods known in the medical arts.

In certain embodiments, the library of compounds to be tested comprises novel compounds. In some embodiments, the library of compounds comprise FDA-approved drugs. In certain embodiments, the library of compounds comprises FDA-approved drugs approved for the treatment of diseases other than ocular cancer. In certain embodiments, the library of compounds comprises natural products and their derivatives; synthetic and natural toxic substances; inhibitors of DNA/RNA synthesis, protein synthesis, cellular respiration, and membrane integrity; and classical and experimental pesticides, herbicides, and endocrine disruptors. In some embodiments, the library of compounds comprises alkaloids, sesquiterpenes, diterpenes, penacyclic triterpenes, and/or sterols. In certain embodiments, the library of compounds comprises cardenolides. Cardenolides may be of the formula:

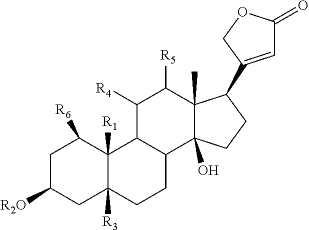

In some embodiments, the library of compounds comprise cardiac glycosides. In some embodiments, the library of compounds comprise compounds with molecular structures related to digoxin. In some embodiments, the library of compounds comprise compounds with molecular structures related to ouabain.

Any cells including normal and abnormal cells may be used in the inventive methods. Cells used in the inventive method may be ocular cancer cells. In some embodiments, the cells used in the inventive method are retinoblastoma cells. In certain embodiments, the cells are human retinoblastoma cells such as, for instance, Y79, WERI-Rb-1, and RB355 cells. In certain embodiments, the cells utilized in the inventive method are a luciferase-expressing Y79LUC cell line. In some embodiments, the cells utilized in the inventive method are human uveal melanoma C918 cells. In some embodiments, the cells utilized in the inventive method are human uveal melanoma Mum2b cells.

In certain embodiments, the cells are incubated with a test compound for approximately 1 minute to approximately 1 week. In certain embodiments, the cells are incubated with a test compound for approximately 1 hour to approximately 1 week. In certain embodiments, the cells are incubated with a test compound for approximately 12 hours to approximately 1 week. In certain embodiments, the cells are incubated with a test compound for approximately 24 hours to approximately 1 week. In certain embodiments, the cells are incubated with a test compound for approximately 36 hours to approximately 1 week. In certain embodiments, the cells are incubated with a test compound for approximately 48 hours to approximately 1 week. In certain embodiments, the cells are incubated with a test compound for approximately 48 hours to approximately 120 hours. In certain embodiments, the cells are incubated with a test compound for approximately 48 hours to approximately 96 hours. In certain embodiments, the cells are incubated with a test compound for approximately 62 hours to approximately 82 hours. In certain embodiments, the cells are incubated with a test compound for approximately 72 hours. In certain embodiments, the cells are incubated with a test compound for 1, 2, 3, 4, 5, 6, or 7 days.

In some embodiments, the instant invention provides a method of determining dose response using a cytotoxicity assay. In some embodiments, the instant invention includes a method of performing dose response studies comprising the steps of providing a library of test compounds, contacting said library of test compounds with a cell, and incubating the cell with the compound under suitable conditions to determine the cytotoxicity of the compounds. The antiproliferative activity of the test compounds can then be assessed using a method known to those of ordinary skill in the art. This process can then be repeated using different concentrations of test compounds in order to calculate the IC₅₀. In certain embodiments, the test compounds are cardenolides. In certain embodiments, the cells are retinoblastoma cells.

In certain embodiments, the cells are incubated with a test compound for approximately 1 minute to approximately 1 week. In certain embodiments, the cells are incubated with a test compound for approximately 1 hour to approximately 1 week. In certain embodiments, the cells are incubated with a test compound for approximately 12 hours to approximately 1 week. In certain embodiments, the cells are incubated with a test compound for approximately 24 hours to approximately 1 week. In certain embodiments, the cells are incubated with a test compound for approximately 36 hours to approximately 1 week. In certain embodiments, the cells are incubated with a test compound for approximately 48 hours to approximately 1 week. In certain embodiments, the cells are incubated with a test compound for approximately 48 hours to approximately 120 hours. In certain embodiments, the cells are incubated with a test compound for approximately 48 hours to approximately 96 hours. In certain embodiments, the cells are incubated with a test compound for approximately 62 hours to approximately 82 hours. In certain embodiments, the cells are incubated with a test compound for approximately 72 hours. In certain embodiments, the cells are incubated with a test compound for approximately 1, 2, 3, 4, 5, 6, or 7 days.

In certain embodiments, after a specified amount of time (e.g., 72 hours) an indicator of cell viability (e.g., Alamar Blue) is added, and the mixture is incubated for an additional period of time. In some embodiments, this additional period of time ranges from approximately 1 hour to approximately 48 hours. In some embodiments, this additional period of time ranges from approximately 12 hour to approximately 36 hours. In some embodiments, this additional period of time is approximately 24 hours.

The inhibition of cell proliferation may be measured using methods or technology known in the art. In some embodiments, inhibition of cell proliferation is measured using a substance which produces a detectable signal that is proportional to the amount of inhibition of cell proliferation. In some embodiments, inhibition of cell proliferation is quantified using one of any indicators known to those of ordinary skill in the art that produces a quantifiable signal, the intensity of which is detectable and proportional to the amount of inhibition. In some embodiments, inhibition of cell proliferation is quantified using an indicator which fluoresces. Exemplary indicators include Tyramide-Alexa Fluor 488, Alamar Blue, etc.

As detailed herein, in assays to determine the ability of a compound (e.g., cardenolide) to inhibit cancer cell growth certain compounds may exhibit IC₅₀ values ≦100 μM. In certain other embodiments, inventive compounds exhibit IC₅₀ values ≦50 μM. In certain other embodiments, inventive compounds exhibit IC₅₀ values ≦40 μM. In certain other embodiments, inventive compounds exhibit IC₅₀ values ≦30 μM. In certain other embodiments, inventive compounds exhibit IC₅₀ values ≦20 μM. In certain other embodiments, inventive compounds exhibit IC₅₀ values ≦10 μM. In certain other embodiments, inventive compounds exhibit IC₅₀ values ≦7.5 μM. In certain embodiments, inventive compounds exhibit IC₅₀ values ≦5 μM. In certain other embodiments, inventive compounds exhibit IC₅₀ values ≦2.5 μM. In certain embodiments, inventive compounds exhibit IC₅₀ values ≦1 μM. In certain embodiments, inventive compounds exhibit IC₅₀ values ≦0.75 μM. In certain embodiments, inventive compounds exhibit IC₅₀ values ≦0.5 μM. In certain embodiments, inventive compounds exhibit IC₅₀ values ≦0.25 μM. In certain embodiments, inventive compounds exhibit IC₅₀ values ≦0.1 μM. In certain other embodiments, inventive compounds exhibit IC₅₀ values ≦75 nM. In certain other embodiments, inventive compounds exhibit IC₅₀ values ≦50 nM. In certain other embodiments, inventive compounds exhibit IC₅₀ values ≦25 nM. In certain other embodiments, inventive compounds exhibit IC₅₀ values ≦10 nM. In other embodiments, exemplary compounds exhibited IC₅₀ values ≦7.5 nM. In other embodiments, exemplary compounds exhibited IC₅₀ values ≦5 nM.

### Method of Inducing Apoptosis

The instant invention also provides methods of inducing apoptosis comprising contacting a cell with a cardenolide in an effective amount to induce apoptosis. In some embodiments, the instant invention provides a method of inducing apoptosis comprising the steps of first providing a cardenolide, contacting the cardenolide with a cell, and then incubating the cells under suitable conditions to induce apoptosis. The extent of apoptosis can be assessed using methods known to detect cells undergoing apoptosis.

In certain embodiments, the cell is an ocular cancer cell. In certain embodiments, the cell is a retinoblastoma cell. In certain embodiments, the cell is derived from a human retinoblastoma cell line such as Y79, WERI-Rb-1, RB355, and Y79LUC. In certain embodiments, the cell is derived from a melanoma cell line such as a uveal melanoma cell line. In certain embodiments, the uveal melanoma cell line is C918 or Mum2b. In certain embodiments, the cardenolide digoxin, acetyldigoxins, ouabain, neriifolin, digoxigenin, peruvoside, digitoxin, acetyldigitoxins, digitoxigenin, medigoxin, strophanthins, cymarin, and strophanthidin.

The method may further comprise administering a second chemotherapeutic agent, for instance, carboplatin, etoposide, teniposide, or verapamil to the cell. In some embodiments, the second chemotherapeutic agent is vincristine, calcitriol, melphalan, 5-fluorouracil, cyclosporin, cisplatin, or Nutlin-3. In certain embodiments, the second chemotherapeutic is an inhibitor of MDR1/Pgp. In some embodiments, the second chemotherapeutic agent is a multidrug resistance-associated protein-1 (MRP1) inhibitor. In some embodiments, the second chemotherapeutic agent is an ABC transporter inhibitor.

The extent of apoptosis may be measured using an indicator of apoptosis. In some embodiments, the extent of apoptosis is quantified using one of any indicators known to those of ordinary skill in the art that produces a quantifiable signal, the intensity of which is detectable and proportional to the extent of apoptosis. In some embodiments, the extent of apoptosis is determined using an indicator medium which fluoresces. In certain embodiments, the extent of apoptosis is determined using immunofluorescence detection of cleaved Caspase-3. In some embodiments, the extent of apoptosis is determined using nuclear staining techniques.

### In Vivo Studies

The instant invention also provides a method of assaying in vivo the efficacy of a compound against tumor cells by providing a therapeutically effective amount of a test compound in a composition suitable for administration to a host animal with a tumor, administering said composition to a host animal with a tumor, and lastly assessing the antitumor effect of the test compound by monitoring the tumor over a period of time.

In some embodiments, the tumors are artificially implanted tumors. In some embodiments, the tumors are xenografts. In some embodiments, the tumors are xenografts comprising cells selected from any one of the group consisting of human retinoblastoma cells Y79, WERI-Rb-1, RB355, and Y79LUC. In some embodiments, the tumors are xenografts comprising Y79LUC cells embedded in matrigel. In some embodiments, the tumors are xenografts comprising cells selected from any one of the group consisting of human uveal melanoma cells lines C918 and Mum2b.

Xenograft tumors are typically grown in the host animal to a certain size prior to administration of the test compound. In some embodiments, xenografts are grown to a size ranging from approximately 50 to approximately 500 mm³. In some embodiments, xenografts are grown to a size ranging from approximately 100 to approximately 400 mm³. In some embodiments, xenografts are grown to a size ranging from approximately 200 to approximately 300 mm³. In some embodiments, xenografts are allowed to reach a size of at least approximately 250 mm³ prior to administration of the test compound.

The method comprises administration of the test compounds in a therapeutically effective dose to the host animal. In some embodiments, a therapeutically effective dose comprises an amount ranging from approximately 0.1 mg/kg to approximately 50.0 mg/kg. In some embodiments, a therapeutically effective dose comprises an amount ranging from approximately 0.5 mg/kg to approximately 50.0 mg/kg. In some embodiments, a therapeutically effective dose comprises an amount ranging from approximately 0.5 mg/kg to approximately 40.0 mg/kg. In some embodiments, a therapeutically effective dose comprises an amount ranging from approximately 0.5 mg/kg to approximately 30.0 mg/kg. In some embodiments, a therapeutically effective dose comprises an amount ranging from approximately 1.0 mg/kg to approximately 25.0 mg/kg. In some embodiments, a therapeutically effective dose comprises an amount ranging from approximately 1.5 mg/kg to approximately 15.0 mg/kg. In some embodiments, treatment is administered locally. In some embodiments, treatment is administered by continuous infusion over a certain period of time. In certain embodiments, administration is via intraarterial infusion. In certain embodiments, administration is via intraarterial infusion via an artery feeding the tumor being treated. In some embodiments, when treatment of the eye is desired, intraarterial infusion occurs via the ophthalmic artery of the eye of the host animal.

In some embodiments, the efficacy of the test compounds is measured by measuring tumor size over a period of time before, during, and/or after treatment with the test compounds. In some embodiments, the test compound is a cardenolide such as, for instance, digoxin. In some embodiments, tumor size is measured once a week. In some embodiments, tumor size is measured twice a week. In some embodiments, tumor size is measured daily. In some embodiments, tumor size is measured once a day. In some embodiments, tumor size is measured twice a day. In some embodiments, tumor size is measured once every other day. In some embodiments, tumor size is measured once every three days. In certain embodiments, tumor size is measured at intervals as required by any one of the methods known to those of skill in the art. In some embodiments, tumor size is measured externally twice a week with a caliper. In certain embodiments, tumor size is measured once a week using an imaging technique (e.g., MRI, X-ray, CT). In some embodiments, the imaging technique is bioluminescent imaging. In certain embodiments, bioluminescent imaging comprises anesthetization of the host animal, injection of a bioluminescent compound, and subsequent measurement of photonic emission. In some embodiments, imaging of the tumor is achieved using any of the methods known in the medical arts.

A subject may be any animal. In certain embodiments, the subject is any mammal (e.g., humans, domestic/veternary/farm animals such as dogs, cats, cows, sheep, etc.). In some embodiments, the subject is a rodent. In certain embodiments, the subject is a human (e.g., child, juvenile, adult, male, female). In certain embodiments, the subject is an experimental animal such as a mouse, rat, dog, or non-human primate.

A therapeutically effective amount of a compound comprises administering an amount necessary to achieve a desired result. The exact amount required will vary from subject to subject, depending on the species, age, general condition of the subject, the severity of the disease, the particular anticancer agent, its mode of administration, the desired outcome, the xenograft, and the like.

In certain embodiments of the present invention a “therapeutically effective amount” of the compound or pharmaceutical composition is that amount effective for inhibiting cell proliferation in a subject or a biological sample (e.g., in cells). In certain embodiments, cell proliferation is inhibited by about 10%, about 20%, about 30%, about 40%, about 50%, about 60%, about 70%, about 80%, about 90%, about 95%, or about 99%. In certain embodiments, the compound inhibits cell proliferation by at least about 25%, at least about 50%, at least about 75%, or at least about 90%.

In certain embodiments of the present invention, a “therapeutically effective amount” refers to an amount of a compound or composition sufficient to inhibit cell proliferation, or refers to an amount of a compound or composition sufficient to reduce the tumor burden in a subject. In certain embodiments, the tumor burden is reduced by about 1%, about 5%, about 10%, about 20%, about 30%, about 40%, about 50%, about 60%, about 70%, about 80%, about 90%, about 95%, or about 99%. In certain embodiments, the tumor burden is reduced by at least about 25%, at least about 50%, at least about 75%, or at least about 90%.

In certain embodiments of the present invention a “therapeutically effective amount” of the inventive compound or pharmaceutical composition is that amount effective for reducing or inhibiting the growth of tumor cells and/or killing tumor cells.

### Pharmaceutical Compositions

In another aspect, the present invention provides pharmaceutical compositions comprising a cardenolide, or a pharmaceutically acceptable form thereof, and a pharmaceutically acceptable excipient for treatment of an ophthalmic condition (e.g., ocular cancer). In certain embodiments, a therapeutically effective amount of the cardenolide for the treatment of an ocular cancer is included in the pharmaceutical composition. In some embodiments, the cancer being treated is a hematopoietic cancer, a liposarcoma, a lung cancer, a brain cancer, a liver cancer, a pancreatic cancer, or an ocular cancer. In certain embodiments, the cancer is retinoblastoma, medulloepithelioma, ocular melanoma, or lymphoma.

It will also be appreciated that the cardenolide can exist in free form for treatment, or where appropriate, as a pharmaceutically acceptable form thereof. According to the present invention, a pharmaceutically acceptable form includes, but is not limited to, pharmaceutically acceptable salts, esters, salts of such esters, or a prodrug or other adduct or derivative of a cardenolide which upon administration to a subject in need is capable of providing, directly or indirectly, a compound as otherwise described herein, or a metabolite thereof.

As described above, the pharmaceutical compositions of the present invention comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with a cardenolide, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose, and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Injectable preparations, for example, sterile injectable aqueous or oleaginous may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland or fixed oil can be employed including synthetic mono- or diglycerides. In addition, fatty acids such as oleic acid are used in the preparation of injectables.

The cardenolides are preferably formulated in dosage unit form for ease of administration and uniformity of dosage. The expression “dosage unit form” as used herein refers to a physically discrete unit of therapeutic agent appropriate for the subject to be treated. It will be understood, however, that the total daily usage of the compounds and compositions of the present invention will be decided by the attending physician within the scope of sound medical judgment. The specific therapeutically effective dose level for any particular subject or organism will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, The Pharmacological Basis of Therapeutics, Tenth Edition, A. Gilman, J. Hardman and L. Limbird, eds., McGraw-Hill Press, 155-173, 2001, which is incorporated herein by reference in its entirety).

Furthermore, after formulation with an appropriate pharmaceutically acceptable excipient in a desired dosage, the pharmaceutical compositions of this invention can be administered to humans and other animals

In certain embodiments, the compounds of the invention may be administered at dosage levels of approximately 0.001 mg/kg to approximately 50 mg/kg, from approximately 0.01 mg/kg to approximately 25 mg/kg, or from approximately 0.1 mg/kg to approximately 10 mg/kg of subject body weight per day, one or more times a day, to obtain the desired therapeutic effect. It will also be appreciated that dosages smaller than approximately 0.001 mg/kg or greater than approximately 50 mg/kg can be administered to a subject.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutic agents. The particular combination of therapies (e.g., chemotherapy with any one of the compounds mentioned herein, radiation therapy, cryotherapy, brachytherapy, etc.) to be employed in a combination regimen will take into account compatibility of the desired therapeutics and/or therapies and the desired therapeutic effect to be achieved. It will also be appreciated that the therapies employed may achieve a desired effect for the same disorder (for example, an inventive compound may be administered concurrently with another anticancer agent, or they may achieve different effects (e.g., control of any adverse effects).

For example, other therapies or anticancer agents that may be used in combination with the cardenolides include surgery, radiotherapy (e.g., γ-radiation, neutron beam radiotherapy, electron beam radiotherapy, proton therapy, brachytherapy, and systemic radioactive isotopes, etc.), endocrine therapy, biologic response modifiers (e.g., interferons, interleukins, and tumor necrosis factor (TNF)), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/druglistframe).

In certain embodiments, the pharmaceutical compositions of the present invention further comprise one or more additional therapeutic agents (e.g., chemotherapeutic and/or palliative agents). For example, additional therapeutic agents for conjoint administration or inclusion in a pharmaceutical composition with a cardenolide may be an approved chemotherapeutic agent and/or pallative agent, or it may be any one of a number of agents undergoing approval by the Food and Drug Administration. For purposes of the invention, the term “palliative” refers to treatment that is focused on the relief of symptoms of a disease and/or side effects of a therapeutic regimen, but is not curative. For example, palliative treatment encompasses painkillers and antinausea medications. In addition, chemotherapy, radiotherapy and surgery can all be used palliatively (that is, to reduce symptoms without going for cure; e.g., for shrinking tumors and reducing pressure, bleeding, pain and/or other symptoms or signs of cancer).

### EXAMPLES

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise indicated, the entire contents of each of the references cited herein are incorporated herein by reference to help illustrate the state of the art. The following examples contain important additional information, exemplification and guidance which can be adapted to the practice of this invention in its various embodiments and the equivalents thereof.

Cell lines and tissue culture. The human retinoblastoma cell lines Y79 and WERI-Rb-1 were purchased from from the American Type Culture Collection (Manassas, Va.). The human retinoblastoma cell line RB355 originally established by Dr. Brenda Gallie (University of Toronto) and the luciferase-expressing Y79LUC cell line were kindly provided by Dr. Michael Dyer (Saint Jude Children's Research Hospital). The human uveal melanoma cell lines C918 and Mum2b originally established by Dr. Mary Hendrix (University of Iowa) were generously provided by Dr. Daniel Albert (University of Wisconsin). The cell lines Y79, WERI-Rb-1 and RB355 were grown in RPMI 1640 (Invitrogen, Carlsbad, Calif.) with 20% (v/v) fetal bovine serum (Omega Scientific, Tarzana, Calif.), 1% (v/v) penicillin-streptomycin (Gemini Bio-Products, Sacramento, Calif.), 2 mM glutamine (Invitrogen, Carlsbad, Calif.), 1 mM sodium pyruvate (Invitrogen, Carlsbad, Calif.), 4.5 g/L glucose (Invitrogen, Carlsbad, Calif.). The cell line C918 was cultured in DMEM with 10% (v/v) fetal bovine serum (Omega Scientific, Tarzana, Calif.), and 1% (v/v) penicillin-streptomycin. All cell lines were grown under an atmosphere of 5% CO₂ 95% air at 37° C. under 85% humidity.

### Example 1

Cytotoxicity assay for screening in 1536-well microtiter plates. Library compounds were pre-plated in 1 μL of 1% DMSO (v/v) into 1536-well microtiter plates (#3893, Corning Inc., Corning, N.Y.) using a TPS-384 Total Pipetting Solution (Apricot Designs. Monrovia, Calif.). Cells were added in 8 μL medium to the screening plates using a Flexdrop IV (Perkin Elmer, Waltham, Mass.). After 72 h incubation was added 1 μL Alamar Blue using Flexdrop. The cells were then incubated for another 24 h, and the fluorescence intensity was read on the Amersham LEADseeker™ Multimodality Imaging System equipped with Cy3 excitation and excitation filters and FLINT epi-mirror. The signal inhibition induced by the compounds was expressed as a percentage compared to high and low controls located on the same plate, as defined as % Inhibition=(high control average−read value)/(high control average−low control average)×100.

### Example 2

Cytotoxicity assay for dose response in 384-well microtiter plates. Dose response studies were performed in 384-well microtiter plates (#3712, Corning Inc., Corning, N.Y.) according to the following protocol: cells were added in 45 μL medium to the screening plates using Flexdrop. After 72 h incubation was added 5 μL Alamar Blue using Flexdrop. The cells were then incubated for another 24 h, and the fluorescence intensity was read on LEADseeker™ Multimodality Imaging System as previously described. To calculate the IC₅₀ for each compound toward each cell line, the dose response was assessed in duplicate and using 12 point doubling dilutions with 100 μM compound concentration as the upper limit. The dose response curve for each set of data was fitted separately, and the two IC₅₀ values obtained were averaged. For compounds having an IC₅₀ below 1 μM or 0.1 μM, the dose response study was repeated using dilutions starting at 10 μM or 1 μM for more accurate determination of the IC₅₀ value.

Automation System & Screening Data Management. The assays were performed on a fully automated linear track robotic platform (CRS F3 Robot System, Thermo Electron, Canada) using several integrated peripherals for plate handling, liquid dispensing, and fluorescence detection. Screening data files from the Amersham LEADseeker™ Multimodality Imaging System were loaded into the HTS Core Screening Data Management System, a custom built suite of modules for compound registration, plating, data management, and powered by ChemAxon Cheminformatic tools (ChemAxon, Hungary).

Chemical Libraries, Automation System & Screening Data Management. The library used for the pilot screen combines 2,640 chemicals obtained commercially from Prestwick and MicroSource. The MicroSource Library contains 2,000 biologically active and structurally diverse compounds from known drugs, experimental bioactives, and pure natural products. The library includes a reference collection of 160 synthetic and natural toxic substances (inhibitors of DNA/RNA synthesis, protein synthesis, cellular respiration, and membrane integrity), a collection of 80 compounds representing classical and experimental, pesticides, herbicides, and endocrine disruptors, a unique collection of 720 natural products and their derivatives. The collection includes simple and complex oxygen heterocycles, alkaloids, sequiterpenes, diterpenes, pentercyclic triterpenes, sterols, and many other diverse representatives. The Prestwick Chemical Library is a unique collection of 640 high purity chemical compounds, all off patent and carefully selected for structural diversity and broad spectrum, covering several therapeutic areas from neuropsychiatry to cardiology, immunology, anti-inflammatory, analgesia and more, with known safety, and bioavailability in humans. The library is constituted of 90% of marketed drugs and 10% bioactive alkaloids or related substances.

### Example 3

Apoptosis assay. Y79 cells seeded in culture medium in a 24-well plate were treated with either vincristine, etoposide, or ouabain at various concentrations in 1% DMSO (v/v) or with 1% DMSO (v/v) alone as a carrier control for 48 h or 72 h. After a wash in PBS, cells were fixed in solution in 4% (v/v) in PBS for 10 minutes. After a wash in PBS, cells for each condition were dried on a glass slide and washed once with water.

The immunofluorescence detection of cleaved Caspase-3 was performed at the Memorial Sloan-Kettering Cancer Center Molecular Cytology Core Facility using a Discovery XT processor (Ventana Medical Systems, Tucson, Ariz.). A rabbit polyclonal Cleaved Caspase 3 (Asp 175) antibody (#9661 L, Cell Signaling, Danvers, Mass.) was used at a concentration of 0.1 μg/ml. Cells were blocked for 30 minutes in 10% (v/v) normal goat serum, 2% (v/v) BSA in PBS prior to incubation with the primary antibody for 3 hours and subsequent 20 minutes incubation with biotinylated goat anti-rabbit IgG (#PK6101, Vector labs, Burlingame, Calif.) diluted 1:200. The detection was performed with Secondary Antibody Blocker, Blocker D, Streptavidin-HRP D (Ventana Medical Systems, Tucson, Ariz.), followed by incubation with Tyramide-Alexa Fluor 488 (#T20922, Invitrogen, Carlsbad, Calif.). Nuclear staining was then performed by incubating the slides for 15 minutes in a 8 μM Hoechst 33342 (Molecular Probes, Eugene, Oreg.) solution in PBS and washing once with PBS. Automated fluorescence imaging of the green channel (activated Caspase-3) and blue channel (nuclei) was performed using an IN Cell Analyzer 1000 (GE Healthcare).

### Example 4

In vivo studies. Subcutaneous xenograft experiments were performed at the Memorial Sloan-Kettering Cancer Center Antitumor Assessment Core Facility. Y79LUC cells (10E6) embedded in matrigel (BD Biosciences, San Jose, Calif.) were injected subcutaneously in the right flank of 8-weeks old ICR/SCID male mice. Treatment started when the tumors reached approximately 250 mm³. The mice were randomized into three groups and two mice per group were treated with either 10% DMSO (v/v) (control group), 1.5 mg/kg ouabain or 15 mg/kg ouabain. Treatment was performed by continuous subcutaneous infusion using osmotic minipumps (#1007D, Alzet, Cupertino, Calif.). Tumor size was measured externally two times a week with a caliper. Body weight of each mouse was monitored as well as other signs of toxicity throughout the treatment period. Bioluminescent imaging of the tumors was performed once a week and prior to sacrifice as follows: mice were anesthetized by isofluorane inhalation and injected with D-luciferin at 50 mg/Kg (Xenogen) intraperitoneally; photonic emission was measured with the In Vivo Imaging System (IVIS 200, Xenogen) with a collection time of 5 seconds.

### Example 5

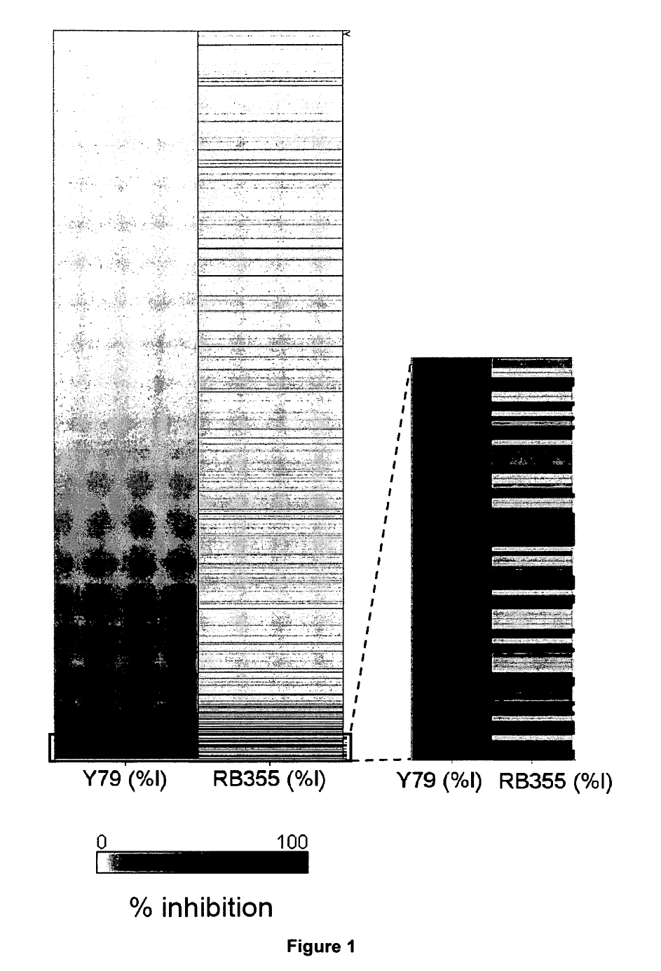

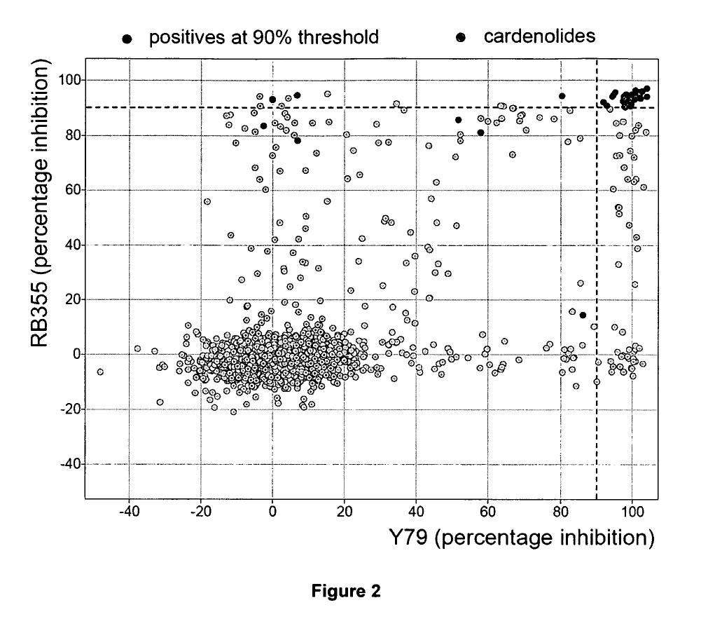

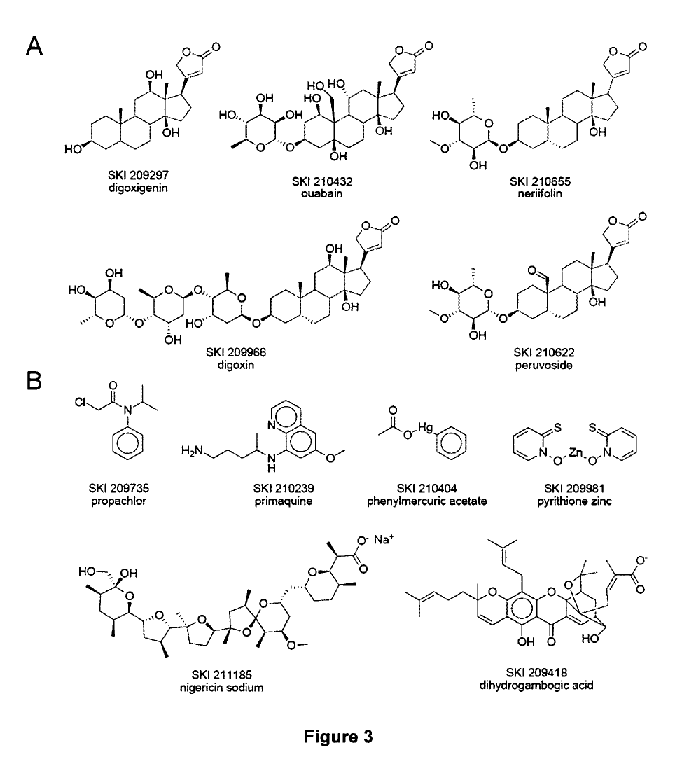

Identification of alternative cytotoxic agents for retinoblastoma among known drugs. The aim was to identify alternative cytotoxic agents for retinoblastoma among known drugs and bioactive agents. To meet this goal, a combined library of 2,640 commercially-obtained chemicals representing biologically active and structurally diverse compounds from known drugs, experimental bioactives, and pure natural products, were screened, most of which were off-patent. For the screen, the use of the well-described cytotoxicity assay based on the reduction of the dye resazurin and commercially sold as Alamar Blue 28 (Ahmed et al., J. Immunol. Methods 1994, 170, 211-224) was relied on due to its compatibility with the requirements of high-throughput screening (Shum et al., J. Enzyme Inhib. Med. Chem. 2008, 1) In this assay, the fluorescence emitted by the living cells upon metabolism of Alamar Blue is proportional to the number of metabolically active cells. Hence, the cytotoxicity or the cytostaticity of a compound can be assessed relative to a control. In an effort to identify chemical scaffolds with broad activity for retinoblastoma as opposed to compounds only cytotoxic toward one specific retinoblastoma cell line, a strategy was adopted to screen the combined drug library in parallel against two retinoblastoma cell lines. Y79 (Reid et al., J. Natl. Cancer Inst. 1974, 53, 347-360) and the RB355 (Fournier et al., Invest. Ophthalmol. Vis. Sci. 1987, 28, 690-699) human cell lines were chosen as models of retinoblastoma because they are among the few well-established human retinoblastoma cell lines available and because it was possible to optimize their growth in high density format. Duplicate sets of the combined library of 2,640 compounds were tested at 10 μM consecutively the same day for each cell line. After statistical analysis of the duplicate sets of data to assess the reproducibility of the screen and to ensure the absence of systematic error, it was calculated the average percentage inhibition for each compound based on high and low controls present on each plate as previously described (Antczak et al., J. Biomol. Screen 2007, 12, 521-535). When the newly generated Y79 and RB355 data sets were compared, it was found that a large population of the tested compounds was active only toward one of the two cell lines (FIG. 1). Next, the percentage inhibition for each compound in both the Y79 and the RB355 data sets (FIG. 2) was compared in a scatter plot. Most tested compounds had no significant activity in either screen, or were active only in one screen. Focus was placed on the population of compounds demonstrating greater than 95% inhibition in both screens in order to select as positives only those compounds that were likely to have broad activity for retinoblastoma. The chemical structures of the selected 11 positives at 95% inhibition threshold are depicted in FIG. 3.

Cytotoxicity profiling was performed for these 11 positives against the human retinoblastoma cell lines Y79, RB355 and WERI-Rb-1, as well as against the uveal melanoma cell lines C918 and Mum2b. It was found that all 11 selected positives had broad and potent cytotoxic activity against these five ocular cancer cell lines with calculated IC₅₀s ranging from 40 nM to 27 μM (Table 1). All selected positives were cytotoxic toward at least three out of five cell lines while most of them (9 out of 11) were potent against all tested cell lines (Table 1). Most of the selected positives could be grouped into two well-known pharmacological classes: ion pump effectors (five) and antimicrobial agents (four). The four most potent compounds identified belonged to the pharmacological class of ion pump effectors. Among them was the drug digoxin, which is currently approved by the FDA for the treatment of cardiac arrhythmia and for the prevention of heart failure.

Cardenolides constitute a class of drugs with broad and potent cytotoxic activity toward ocular cancer cells. A structural analysis of the positives identified during the screen revealed that the five ion pump effectors that we previously characterized (Table 1): peruvoside, ouabain; neriifolin, digoxin and digoxigenin all share a common chemical scaffold (FIG. 3A). This scaffold corresponds to the core structure of cardenolides. When a structural search was performed for compounds present in the combined library sharing the same scaffold, 19 cardenolides were identified. All were found to induce greater than 75% inhibition toward at least one cell line during the screen, and constituted 10 out of the 29 positives at a threshold of 90% inhibition in both screens (FIG. 2). In addition, 13 out of 19 cardenolides (68%) present in the combined library induced greater than 50% inhibition in both screens. This observation led to a focus on cardenolides as a new class of antiproliferative agents for retinoblastoma.

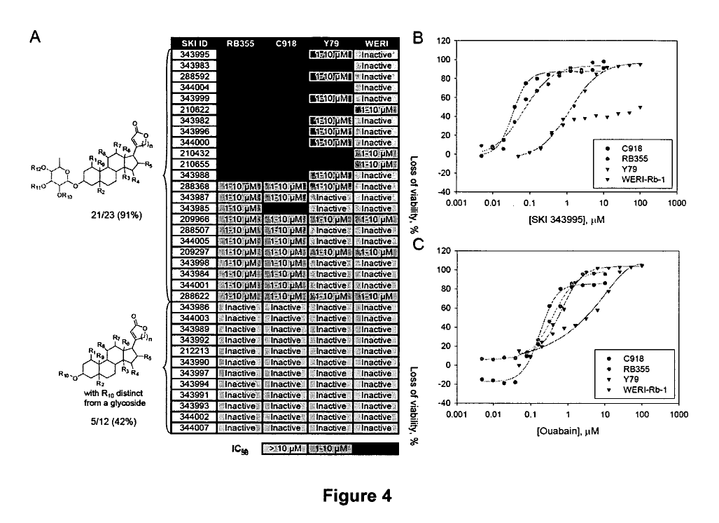

To explore the structure activity relationship (SAR) within this chemical class a collection of 35 cardenolides and derivatives was constituted. The dose response for each compound toward the ocular cancer cell lines Y79, RB355, WERI-Rb-1 and C918 was then assessed. The results of this structure-activity relationship (SAR) study are summarized in FIG. 4. With 23 out of the 35 tested cardenolides (64%) having potent anti-proliferative properties toward at least two ocular cancer cell line tested (IC₅₀<10 μM) (FIG. 4A), it was confirmed that cardenolides constitute a class of potent and broad-acting agents for retinoblastoma. The most potent compound among the 35 tested cardenolides (SKI 343995) had a calculated IC₅₀ of 35 and 90 nM toward the cell lines C918 and RB355, respectively (FIGS. 4A and 4B). As the structure activity relationships underlying the potency of cardenolides in the panel of ocular cancer cells was investigated, a clear trend was identified among the 35 derivatives tested: 21 compounds among the 23 most potent derivatives tested (91%) had a glycoside moiety grafted to their 3-hydroxy group (FIG. 4A). On the other hand, a significant proportion of the 12 less potent compounds (42%) did not have any glycoside moiety at this position (FIG. 4A). Several cardenolides had potent activity across the entire panel of ocular cell lines tested, such as the drug ouabain, which has a long history in the treatment of heart failure (Schoner et al., Am. J. Physiol. Cell Physiol. 2007, 293, C509-536; Rahimtoola et al., Curr. Probl. Cardiol. 1996, 21, 781-853; Newman et al., Mol. Interv. 2008, 8, 36-49) (FIG. 4C).

### Example 6

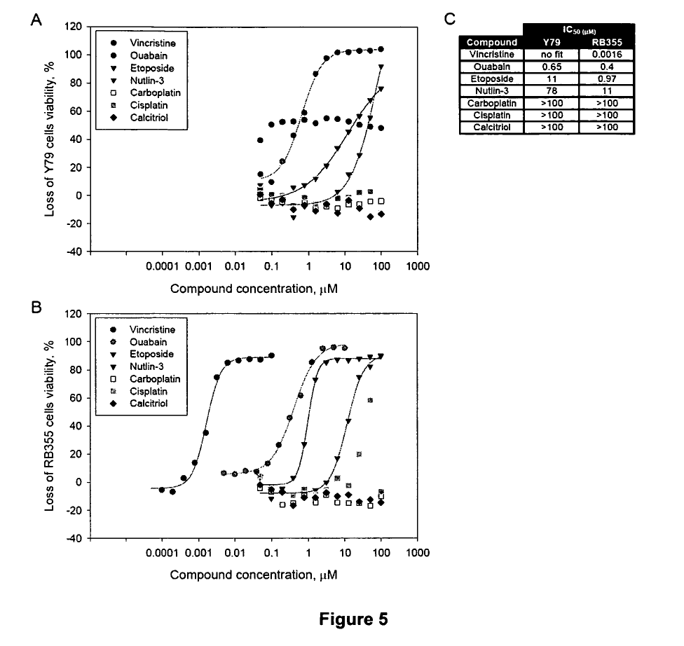

Compared potency of the cardenolide ouabain with known agents in cell models of retinoblastoma. The potency of a representative of the cardenolide scaffold was compared to known effective agents against retinoblastoma. Namely, the dose response of the drug ouabain was tested with the human retinoblastoma cell lines Y79 and RB355, and its potency was compared to vincristine, etoposide, carboplatin, cisplatin, nutlin-3 and calcitriol. Ouabain was chosen as a representative of cardenolides because it demonstrated broad and potent activity toward all the tested cell lines (Table 1, FIGS. 4A and 4B), and because of its long history as a cardiotonic drug. In this assay, ouabain was the most potent compound toward Y79 cells with an IC₅₀ of 0.65 μM compared to 11 μM for etoposide and 78 μM for nutlin-3 (FIG. 5A). The activity of vincristine toward Y79 cells reached a plateau at 50% inhibition, which prevented the IC₅₀ for this compound from being calculated. Carboplatin, cisplatin and calcitriol did not demonstrate any significant activity toward Y79 cells below 100 μM in these assays. Ouabain had a similar potency toward RB355 cells with an IC₅₀ of 0.40 μM compared to 1.6 nM for vincristine, 0.97 μM for etoposide and 11 μM for nutlin-3 (FIG. 5B). Cisplatin reached a maximum of 65% inhibition at 100 μM, and neither caboplatin or calcitriol had any significant activity below 100 μM. These results demonstrate that the in vitro potency of the cardenolide ouabain is comparable to or even greater than the most potent agents for retinoblastoma currently known.

### Example 7

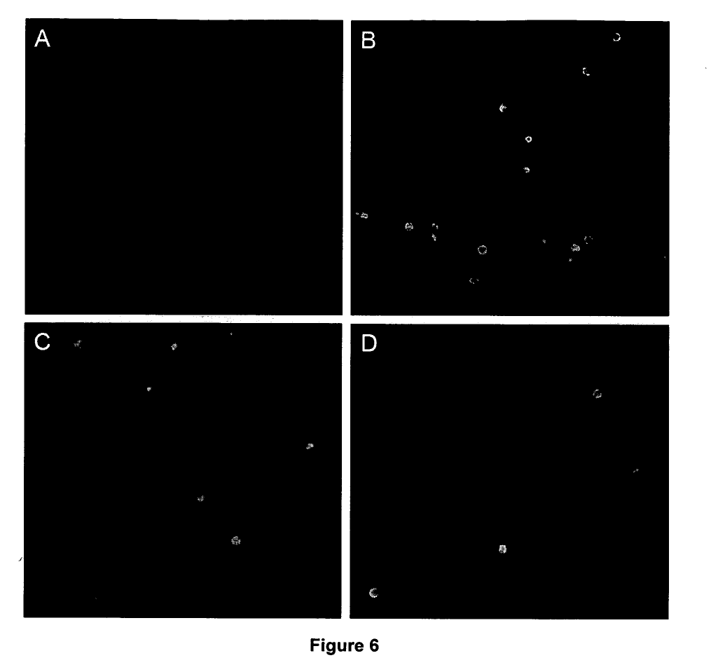

Compared effect of ouabain and clinical agents on apoptosis of Y79 cells. To determine whether the anti-proliferative effects of the drug ouabain was mediated by induction of apoptosis, immunostaining of activated Caspase-3 in Y79 cells treated with cardenolides or known agents for retinoblastoma for 72 h (green channel) was performed; treated cells were also stained with Hoechst to image the nuclei (blue channel) (FIG. 6). The drug concentrations used in this experiment were previously determined according to a pilot study where treated Y79 cells were live-stained with the dye Yo-Pro, which stains apoptic cells (Idziorek et al., J. Immunol. Methods 1995, 185, 249-258). Based on this study, 72 h was identified as the optimum incubation time and drug concentrations were selected that maximized the number of apoptotic cells (data not shown). Baseline Caspase-3 activation was evaluated with control Y79 cells treated with 1% DMSO (v/v) (FIG. 6A). It was found that vincristine (FIG. 6B) and etoposide (FIG. 6C) induced significant apoptosis in Y79 cells compared to baseline levels, as previously described (Elison et al., Arch. Ophthalmol. 2006, 124, 1269-1275; Conway et al., Eur. J. Cancer 1998, 34, 1741-1748; Giuliano et al., Invest. Ophthalmol. Vis. Sci. 1998, 39, 1300-1311). Ouabain in this experiment was used at a concentration of 0.5 μM compared to 100 μM for vincristine and 10 μM for etoposide because higher concentrations of ouabain erradicated Y79 cells in the pilot study with the dye Yo-Pro. At this lower concentration ouabain still induced significant apoptosis (FIG. 6D).

### Example 8

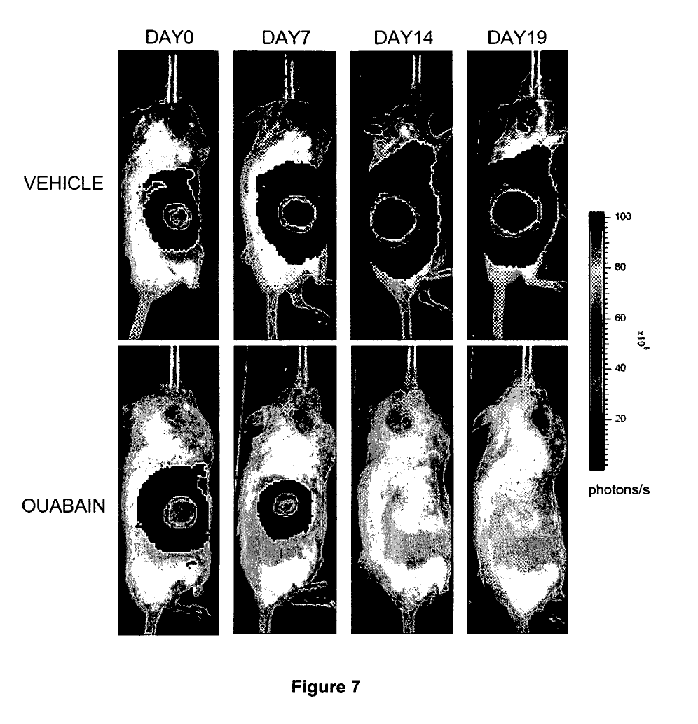

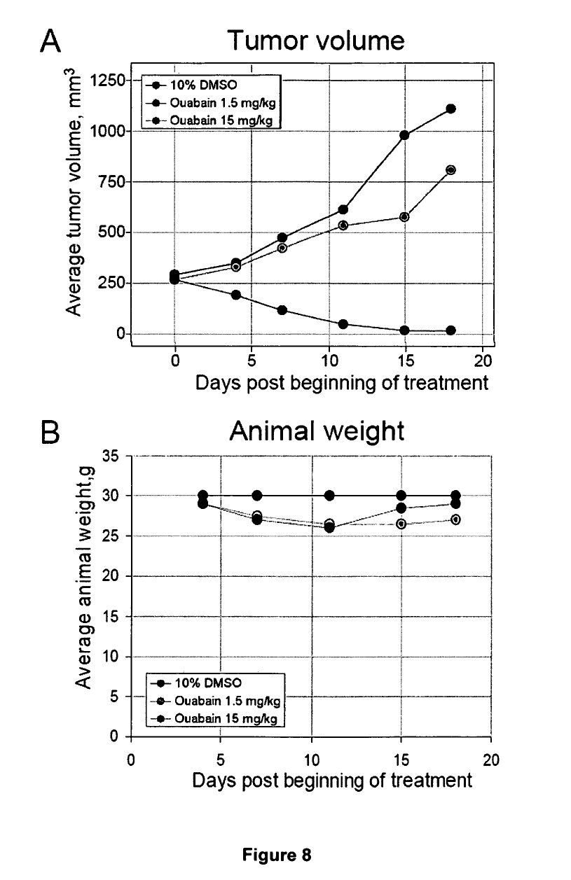

Assessment of the in vivo efficacy of ouabain in a xenograft model of retinoblastoma. The therapeutic effect of the drug ouabain in a mouse xenograft model of retinoblastoma was also investigated. Three groups of two 8 weeks old ICR/SCID male mice bearing Y79 tumors implanted in the flank were treated with either vehicle only, 1.5 mg/kg ouabain or 15 mg/kg ouabain. Mice were continuously infused subcutaneously using an osmotic minipump delivery system. Evaluation of tumor burden by bioluminescent imaging shows that ouabain at 15 mg/kg rapidly induced a dramatic decrease in tumor size leading to complete tumor regression (as assessed by bioluminescence imaging) after 14 days of treatment (FIG. 7). In comparison, tumors in the vehicle-treated control group continuously grew, necessitating to euthanize the animals at day 19. Quantification of tumor size confirmed this result: the average tumor size for the control group reached 1,000 mm³ at day 14 and kept growing while both animals treated with 15 mg/kg ouabain had their tumor nearly eradicated by day 14 (18 mm³ average size) (FIG. 8A). At a lower dose of 1.5 mg/kg, ouabain seemed to reduce the tumor burden compared to the control group (FIG. 8A). Throughout the treatment period, the average body weight of treated and control animals did not differ significantly, indicating that even at the high dose of 15 mg/kg ouabain did not induce any significant toxicity (FIG. 8B).

This research presents an alternative strategy aiming at identifying novel agents for treating retinoblastoma among already approved drugs. Known drugs may have previously unreported antiproliferative properties for retinoblastoma, and could therefore potentially be repositioned as novel drugs for retinoblastoma cells. To investigate this, a combined library of 2,640 marketed drugs and bioactive compounds was compiled and a cytotoxicity assay amenable to high-throughput screening for the human retinoblastoma cell lines Y79 and RB355 was developed. A striking finding of this screening campaign was the discovery of the broad and potent antiproliferative activity toward retinoblastoma cells of the well-described chemical class of cardenolides. This observation was confirmed by establishing basic structure activity relationships (SAR) for a series of 35 cardenolides and derivatives in a panel of four ocular cancer cell lines. When the in vitro antiproliferative properties of the drug ouabain was compared to known or experimental agents for retinoblastoma, it was observed that the potency of ouabain is comparable to agents currently used in clinic. Furthermore, it was demonstrated that the drug ouabain induces apoptosis in Y79 human retinoblastoma cells at a dose of 0.5 μM. This observation is in agreement with previous studies showing that cardenolides induce apoptosis in various cell types (Newman et al., Mol. Interv. 2008, 8, 36-49. Finally, when the therapeutic effect of ouabain was assessed in a xenograft model of retinoblastoma, a drastic response leading to complete tumor regression after 14 days of treatment was observed. Even at the high dose of 15 mg/kg ouabain used in this study, no signs of toxicity were observed.

### Equivalents

Those skilled in the art will recognize, or be able to ascertain using no more than routine experimentation, many equivalents to the specific embodiments of the invention described herein. Such equivalents are intended to be encompassed by the following claims.

1. A method of treating ocular cancer comprising administering a therapeutically effective amount of a cardenolide locally to the eye of a subject with an ocular cancer.

2. The method of claim 1, wherein the cardenolide is of the formula: 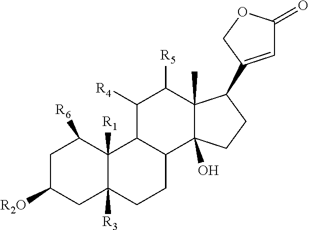
wherein:
R₁ is H, OH, CH₃, CH₂OH, or CHO;
R₂ is H or a carbohydrate moiety;
R₃ is H or OH;
R₄ is H or OH;
R₅ is H or OH;
R₆ is H or OH;
or a pharmaceutically acceptable form thereof.

3. The method of claim 1, comprising administering a cardenolide selected from the group consisting of digoxigenin, ouabain, neriifolin, digoxin, acetyldigoxin, peruvoside, digitoxin, acetyldigitoxins, digitoxigenin, medigoxin, strophanthins, cymarin, and strophanthidin.

4. The method of claim 1, wherein the cardenolide is of one of the formulae:

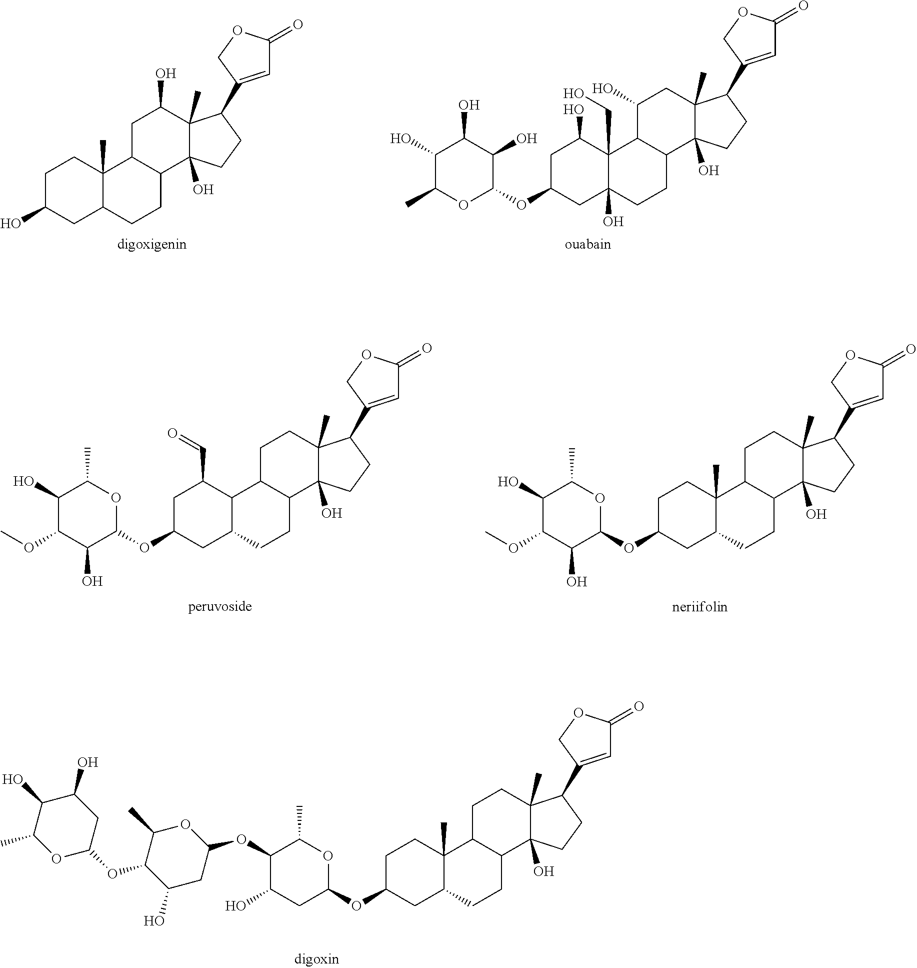

5. (canceled)

6. The method of claim 1, wherein the subject is a mammal.

7-8. (canceled)

9. The method of claim 1, wherein the subject is human.

10. The method of claim 1, wherein the cardenolide is administered via direct intraarterial infusion into the ophthalmic artery.

11. The method of claim 1, wherein the ocular cancer is retinoblastoma.

12. The method of claim 1, wherein the ocular cancer is medulloepithelioma.

13. The method of claim 1, wherein the ocular cancer is ocular melanoma.

14. The method of claim 1, wherein the ocular cancer is lymphoma.

15. The method of claim 1, wherein the cancer has metastasized to the eye.

16. A method of treating ocular cancers comprising administering a therapeutically effective amount of digoxin via direct intraarterial infusion into the ophthalmic artery of the eye of a subject with retinoblastoma.

17. (canceled)

18. The method of claim 16, wherein the subject is a human less than 18 years of age.

19-20. (canceled)

21. The method of claim 1, further comprising administering a second chemotherapeutic agent.

22. The method of claim 1, wherein the second chemotherapeutic agent is selected from the group consisting of cisplatin, carboplatin, etoposide, teniposide, verapamil, vincristine, calcitriol, melphalan, cyclosporin, 5-fluorouracil, topotecan and Nutlin-3.

23. The method of claim 1, further comprising administering radiotherapy, thermotherapy, cryotherapy, or brachytherapy.

24-25. (canceled)

26. A method of inhibiting growth of ocular cancer cells comprising contacting in vitro ocular cancer cells with an effective amount of a cardenolide to inhibit the growth of the cells.

27-33. (canceled)

34. A method of inducing apoptosis comprising contacting a cell with a cardenolide in an amount effective to induce apoptosis.

35-39. (canceled)

40. A pharmaceutical composition for treating an ocular cancer by local administration comprising a cardenolide and a pharmaceutically acceptable excipient.

41. (canceled)
